(19) United States
(12) Patent Application Publication
(10) Pub. No.: US 2002/0081685 A1
(43) Pub. Date: Jun. 27, 2002

(54) Disintegrin homologs, ZSNK10, ZSNK11, and ZSNK12

(76) Inventors: Brian A. Fox, Seattle, WA (US); Paul O. Sheppard, Granite Falls, WA (US)

Correspondence Address: Robyn Adams, ZymoGenetics, Inc., 1201 Eastlake Avenue East, Seattle, WA, 98102, US

(21) Appl. No.: 09/921,823

(22) Filed: Aug. 2, 2001

Related U.S. Application Data
(60) Provisional application No. 60/222,564, filed on Aug. 3, 2000.

(51) Int. Cl. C12N 9/00; C07H 21/04; C12P 21/02; C12N 5/06

(52) U.S. Cl. 435/183; 435/325; 435/320.1; 536/23.2

(57) ABSTRACT

The present invention relates to polynucleotide and polypeptide molecules, and variants thereof, for ZSNK10, ZSNK11, and ZSNK12, novel members of the Disintegrin Proteases. The polypeptides, and polynucleotides encoding them, are cell-cell interaction modulating and may be used for delivery and therapeutics. The present invention also includes antibodies to the ZSNK10, ZSNK11, and ZSNK12 polypeptides.

### REFERENCE TO RELATED APPLICATIONS

This application is related to Provisional Application No. 60/222,564, filed on Aug. 3, 2000. Under 35 U.S.C. §119(e)(1), this application claims benefit of said Provisional Application.

### BACKGROUND OF THE INVENTION

Disintegrins have been shown to bind cell surface molecules, including integrins, on the surface of various cells, such as platelets, fibroblasts, tumor, endothelial, muscle, neuronal, bone, and sperm cells. Disintegrins are unique and potentially useful tools for investigating cell-matrix and cell-cell interactions. Additionally, they have been useful in the development of antithrombotic and antimetastatic agents due to their anti-adhesive, anti-migration of certain tumor cells, and anti-angiogenesis activities.

Families of proteins which have disintegrin domains include ADAMs (A Disintegrin and Metalloprotease), MDCs (Metalloprotease/Disintegrin/Cysteine-rich) and SVMPs (Snake Venom Metalloprotease)., herein termed Disintegrin Protease (DP) protein family.

For a review of ADAMs, see Wolfsberg and White, Developmental Biology, 180:389-401, 1996. ADAMs have been shown to exist as independent functional units as well as in conjunction with other members of this family in heterodimeric complexes. Some members of the family have multiple isoforms which may have resulted from alternative splicing. ADAMs proteins have been shown to have adhesive as well as anti-adhesive functions in their extracellular domains. Some members of the ADAMs family have very specific tissue distribution while others are widely distributed. Not all members of this family are capable of manifesting all of the potential functions represented by the domains common to their genetic structure.

The ADAMs are characterized by having a propeptide domain, a metalloprotease-like domain, a disintegrin-like domain, a cysteine-rich domain, an EGF-like domain, and a cytoplasmic domain.

A prototypical example of this family is ADAM 12. ADAM 12, also known as meltrin a, has a truncated isoform, as well as a full-length isoform, and is involved in muscle cell fusion and differentiation (Gilpin et al., J. Biol. Chem. 273:157-166, 1998). Other ADAMs involved in fusion are ADAM 1, and ADAM 2 which form a heterodimer (fertilin) and are involved in sperm/egg fusion (Wolfsberg and White, supra).

The SVMP family is represented by three classes (P-I, P-II, and P-III). All three classes contain propeptide and metalloprotease domains. The P-II and P-III classes also contain a disintegrin domain, and the P-III class further contains a cysteine-rich domain. These domains are similar in sequence to those found in the ADAMs. Some members of the SVMP family have a conserved “RGD” amino acid sequence. This tripeptide has been shown to form a hairpin loop whose conformation can disrupt the binding of fibrinogen to activated platelets. This “RGD” sequence may be substituted by RSE, MVD, MSE, and KGD in P-II SVMPs, and by MSEC (SEQ ID NO:10), RSEC (SEQ ID NO:11), IDDC (SEQ ID NO:12), and RDDC (SEQ ID NO:13) (a tripeptide along with a carboxy-terminal cysteine residue) in P-III SVMPs. Thus, these sequences may be responsible for integrin binding in the P-II and P-III SVMPs.

A prototypical example of a SVMP is jararhagin, which mediates platelet aggregation by binding to the platelet a₂ subunit (GPIa) via the disintegrin domain followed by proteolysis of the b₁ subunit (GPIIA) (Huang and Liu, J. Toxicol-Toxin Reviews 16: 135-161, 1997).

The proteins of the Metalloprotease/Disintegrin/Cysteine-rich family are involved in diverse tasks, ranging from roles in fertilization and muscle fusion, TNFa release from plasma membranes, intracellular protein cleavage, and essential functions in neuronal development (Blobel, C. P. Cell 90:589-592, 1997). This family is also characterized by the metalloprotease, disintegrin and cysteine-rich domains, as described above.

Members of the DP family of proteins which have been shown to be therapetuically useful include eptifibatide (Integrilin®, made by COR Therapeutics, Inc. and Key Pharmaceuticals, Inc.) which is useful as an anti-clotting agent for acute coronary syndrome, and contortrostatin, which inhibits β₁Integrin-mediated human metastatic melanoma cell adhesion and blocks experimental metastasis (Trikha, M. et at., Cancer Research 54: 4993-4998, 1994) and inhibits platelet aggregation (Clark, E. A. et al., J. Biol. Chem. 269 (35):21940-21943, 1994).

The present invention provides a novel member of the Disintegrin Proteases and related compositions whose uses will be apparent to those skilled in the art from the teachings herein.

### SUMMARY OF THE INVENTION

Within one aspect, the invention provides an isolated polypeptide molecule comprising the amino acid sequence as shown in SEQ ID NO:17 from residue 461 to residue 474. Within an embodiment, the polypeptide molecule comprises the amino acid sequence from residue 398 to residue 492 as shown in SEQ ID NO:17. Within another embodiment, the polypeptide molecule comprises the amino acid sequence from residue 191 to residue 492 as shown in SEQ ID NO:17. Within another embodiment, the polypeptide molecule comprises the amino acid sequence from residue 1 to residue 611 as shown in SEQ ID NO:17. Within another embodiment, the invention provides an isolated polynucleotide molecule encoding the polypeptide molecule comprising amino acid residues 461 to 474 as shown in SEQ ID NO:17. Within another embodiment, the invention provides an isolated polynucleotide molecule encoding the polypeptide molecule comprising amino acid residues 398 to 492 as shown in SEQ ID NO:17. Within another embodiment, the invention provides an isolated polypeptide molecule comprising the amino acid sequence as shown in SEQ ID NO:17 from residue 191 to residue 395. Within another embodiment, the invention provides an isolated polynucleotide molecule encoding the polypeptide molecule comprising the amino acid sequence as shown in SEQ ID NO:17 from residue 191 to residue 395. Within another embodiment, the invention provides an isolated polynucleotide molecule encoding the polypeptide molecule comprising amino acid residues 191 to 492 as shown in SEQ ID NO:17. Within another embodiment, the invention provides an isolated polynucleotide molecule encoding the polypeptide molecule comprising amino acid residues 1 to 611 as shown in SEQ ID NO:17.

Within another aspect, the invention provides an expression vector comprising the following operably linked elements: a) a transcription promoter; b) a DNA segment encoding the polypeptide comprising the amino acid sequence from residue 398 to residue 492 as shown in SEQ ID NO:17; and c) a transcription terminator. Within another embodiment, the DNA segment further encodes an affinity tag. Within another embodiment, the invention provides a cultured cell into which has been introduced the expression vector and the cell expresses the polypeptide encoded by the DNA segment. Within another embodiment, is provided a method of producing a polypeptide comprising culturing the cell, whereby said cell expresses the polypeptide encoded by the DNA segment, and the polypeptide is recovered. Within another embodiment, the polypeptide produced by the method is provided.

Within another aspect, the invention provides a method of producing an antibody comprising the following steps: inoculating an animal with a polypeptide such that the polypeptide elicits an immune response in the animal to produce the antibody; and isolating the antibody from the animal; and wherein the polypeptide comprises at least 15 consecutive amino acids of SEQ ID NO:17. Within another embodiment, the antibody binds to the polypeptide as shown in SEQ ID NO:17. Within another embodiment, the antibody which specifically binds to a polypeptide comprising amino acid residues 1 to 611 of SEQ ID NO:17.

Within another aspect, the invention provides an isolated polypeptide molecule comprising the amino acid sequence as shown in SEQ ID NO:23 from residue 85 to residue 97. Within another embodiment, the polypeptide molecule comprises the amino acid sequence from residue 51 to residue 110 as shown in SEQ ID NO:23. Within another embodiment, the polypeptide molecule comprises the amino acid sequence from residue 1 to residue 110 as shown in SEQ ID NO:23. Within another embodiment, is provided an isolated polynucleotide molecule encoding the polypeptide molecule comprising the amino acid sequence as shown in SEQ ID NO:23 from residue 85 to residue 97. Within another embodiment, the invention provides an expression vector comprising the following operably linked elements: a) a transcription promoter; b) a DNA segment encoding the polypeptide molecule comprising the amino acid sequence from residue 51 to residue 110 as shown in SEQ ID NO:23; and c) a transcription terminator. Within another embodiment, the DNA segment further encodes an affinity tag. Within another embodiment, the invention provides a cultured cell into which has been introduced the expression vector, wherein said cell expresses the polypeptide encoded by the DNA segment. Within another embodiment, the invention provides a method of producing a polypeptide comprising culturing the cell, whereby said cell expresses the polypeptide encoded by the DNA segment, and the polypeptide is recovered. Within another embodiment, is provided the polypeptide produced by the method.

Within another aspect, the invention provides a method of producing an antibody comprising the following steps: inoculating an animal with a polypeptide such that the polypeptide elicits an immune response in the animal to produce the antibody; and isolating the antibody from the animal; and wherein the polypeptide comprises at least 15 consecutive amino acids of SEQ ID NO:23. Within another embodiment, the antibody produced by the method binds to the polypeptide as shown in SEQ ID NO:23. Within another embodiment, the invention provides an antibody which specifically binds to a polypeptide comprising amino acid residues 1 to 110 of SEQ ID NO:23.

Within another aspect, the invention provides an isolated polypeptide molecule comprising the amino acid sequence as shown in SEQ ID NO:20 from residue 191 to residue 393. Within another embodiment, the polypeptide molecule comprises the amino acid sequence from residue I to residue 433 as shown in SEQ ID NO:20. Within another embodiment, is provided an isolated polynucleotide molecule encoding the polypeptide molecule comprising the amino acid sequence as shown in SEQ ID NO:20 from residue 191 to residue 393. Within another embodiment, the invention provides an expression vector comprising the following operably linked elements: a) a transcription promoter; b) a DNA segment encoding the polypeptide molecule; and c) a transcription terminator. Within another embodiment, the DNA segment further encodes an affinity tag. Within another embodiment, the invention provides a cultured cell into which has been introduced the expression vector, wherein said cell expresses the polypeptide encoded by the DNA segment. Within another embodiment, the cell expresses the polypeptide encoded by the DNA segment, and the polypeptide is recovered. Within another embodiment, is provided the polypeptide produced by the method.

Within another aspect, the invention provides a method of producing an antibody comprising the following steps: inoculating an animal with a polypeptide such that the polypeptide elicits an immune response in the animal to produce the antibody; and isolating the antibody from the animal; and wherein the polypeptide comprises at least 15 consecutive amino acids of SEQ ID NO:20. Within another embodiment, the antibody binds to the polypeptide as shown in SEQ ID NO:20. Within another embodiment, the antibody which specifically binds to a polypeptide comprising amino acid residues 1 to 433 of SEQ ID NO:20.

Within another aspect, the invention provides a method of modulating cell-cell interactions comprising contacting the cells with a polypeptide chosen from the group consisting of: a) a polypeptide comprising the amino acid sequence as shown in SEQ ID NO:17 from residue 1 to residue 474; and b) a polypeptide comprising the amino acid sequence as shown in SEQ ID NO:23 from residue 85 to residue 97. Within another embodiment, the invention provides a method of modulating cell-cell interactions comprising contacting the cells with an antibody, wherein the antibody specifically binds to a polypeptide chosen from the group consisting of: a) a polypeptide comprising the amino acid sequence as shown in SEQ ID NO:17 from residue 1 to residue 474; b) a polypeptide comprising the amino acid sequence as shown in SEQ ID NO:20 from residue 1 to residue 433; and c) a polypeptide comprising the amino acid sequence as shown in SEQ ID NO:23 from residue 85 to residue 97.

These and other aspects of the invention will become evident upon reference to the following detailed description of the invention and attached drawings.

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:7), substance P, Flag™ peptide (Hopp et al., Biotechnology 6:1204-1210, 1988), streptavidin binding peptide, maltose binding protein (Guan et al., Gene 67:21-30, 987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “ortholog” or “species homolog”, denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain or multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based upon the discovery of novel cDNA sequences (SEQ ID NOs: 1, 4, 7, 16, 19 , and 22) and corresponding polypeptides having homology to disintegrin-like family members (ADAMs, SVMPs and MDCs; referred to herein as Disintegrin Proteases, or “DPs”). See, for example, Blobel, C. P., Cell 90:589-592, 1997, Jia, J. Biol. Chem. 272:13094-13102, 1997; and Wolfsberg and White, Developmental Biology 180:389-401, 1996. Disintegrins can be involved in, for example, anticoagulation, fertilization, muscle fusion, and neurogenesis. Polynucleotides and polypeptides of the present invention have been designated zsnk10, zsnk11, and zsnk12.

A discussion of the domain structure of some members of the DPs will aid to illustrate the present invention in better detail. The secretory peptide has been described above.

The propeptide domain is usually amino-terminal to the metalloprotease domain and is can act as an inhibitor for the metalloprotease domain (presumably via a cysteine-switch mechanism), such that the metalloprotease domain is activated in certain circumstances. This inhibition can be by blocking the active site of the metalloprotease domain.

The protease domain may be active or inactive. Some members of the disintegrin family have “active” zinc catalytic sites which may be regulated by a “cysteine-switch” in the cysteine-rich domain. Examples of family members which have “active” protease domains are ADAM 1 and ADAM 10, which are involved in sperm/egg fusion and degradation of myelin basic sheath protein, respectively. Members of this family which do not have such a catalytic site include, for example, ADAM 11, which may be involved in tumor suppression. Other protein families which are know to have inactive protease domains are the serine proteases.

The adhesion (disintegrin) domain binds integrins or cell surface receptors which can be located on the surface of a multitude of cells, depending on the specificity of the disintegrin. The predicted binding site within this disintegrin domain is often an amino acid loop comprising about 13 to 14 amino acids. See Wolfsbeg and White, supra) The conformation of this sequence upon folding results in a hairpin loop presenting an amino acid sequence at its tip. This sequence is often “RGD”, but may be substituted by a variety of other amino acid residues, including “XXCD” (SEQ ID NO:14) (Wolfsberg and White, supra; and Jia, J. Biol. Chem. 272:13094-13102, 1997). The diversity of these sequences may reflect that: 1) not all disintegrin domains serve as ligands for integrins (or other cell surface receptors); 2) disintegrin domains with different sequences bind to different types of integrins or cell surface receptors; or 3) the important part of the disintegrin loop is its structure, not its sequence, and thus, that the integrins or receptors for the specific classes of disintegrin domains can recognize a multitude of disintegrin binding loop sequences. Disintegrin domains have been shown to be responsible for cell-cell interactions, including inhibition of platelet aggregation by binding GPIIb/IIIa (fibronectin receptor) and/or GPIa/IIa (collagen receptor).

Many disintegrin family members have a fusion domain, a relatively hydrophobic domain of about 23 amino acids. This domain is present within some of the ADAM family members, and has been shown to be involved in cell-cell fusion, and particularly in sperm/egg fusion, and muscle fusion.

The cysteine-rich domain varies in the DP family members and is believed to be involved in structurally presenting the integrin-binding region to integrins. For the disintegrin-like members of this family, the cysteine-rich domain may also be necessary for secondary structure conformation of the polypeptide, specifically, disulfide bonding between the disintegrin domain and the cysteine domain.

Some members of this group of proteins also contain a thrombospondin-like (TSP-like ) domain that is located at the carboxyl terminal of the protein. Multiple TSP-like domains can be present. For example, METH-1 has three TSP-like domains, and another METH homolog METH-2 (Vasquez, F. et al., J. Biol. Chem. 274: 23349-23357, 1999.) has two TSP-like domains. Thrombospondin-1 is a modular protein that associates with the extracellular matrix and has the ability to inhibit angiogenesis in vivo. Under culture conditions, thrombospondin-1 blocks capillary-like formation and endothelial cell proliferation. Both METH-1 and METH-2 have also been shown to inhibit angiogenesis in the cornea pocket and CAM assays (Vasquez, ibid).

Many DP family members have a transmembrane domain, which acts to anchor the polypeptide to the cell membrane. Membrane-anchored DPs can be involved in a process called “protein ectodomain shedding” wherein the metalloprotease domain cleaves extracellular domain(s) of another protein. In these cases, the metalloprotease can be active on the cell surface itself, as in the case of fertilin (ADAMs 1 and 2), or TACE (ADAM 17), or the metalloprotease can act intracellularly in the secretory pathway as has been described for KUZ and ADAM 10 (Blobel, C. P., supra; and Lammich, S. et al., Proc. Natl. Acad. Sci. USA 96:3922-3927, 1999, respectively). These membrane-anchored metalloproteases are likely to be active in the tissues where their genes are transcribed, in which cases they can be acting in cis, on other proteins bound to the same cell surface, in trans, on proteins bound to other cell surfaces, or on other proteins which are not membrane bound. Additionally the membrane anchor itself can be cleaved resulting in a soluble form of the metalloprotease/disintegrin which can be active at other sites in the body.

The cytoplasmic, or signaling, domain of disintegrin family members tends to be conserved in length and sites for phosphorylation. However, beyond that they tend to be unique in amino acid composition. Some disintegrin family members may signal by binding to the SH3 domain of Abl, Src, and/or Src-related SH3 domains.

The present invention is based upon the discovery of novel domains of three members of the DP family of proteins, designated zsnk10, zsnk11, and zsnk12. The polynucleotide sequences for zsnk10 are shown in SEQ ID NOs: 1, and SEQ ID NO:16. The polypeptide sequences for znsk10 are shown in SEQ ID NO:2 an SEQ ID NO:17. Domains of zsnk10 include: a signal domain, (residue 1 to residue 18 of SEQ ID NO:17); a pro-peptide domain (residue 19 to residue 187 of SEQ ID NO:17); a metalloprotease domain (residue 1 to residue 140 as shown in SEQ ID NO:2; also shown as residue 191 to residue 395 of SEQ ID NO:17), a disintegrin-like domain (residue 141 to residue 137 as shown in SEQ ID NO:2 also shown as residue 398 to residue 492 of SEQ ID NO:17) and a cysteine-rich domain (partial, residue 238 to residue 256 as shown in SEQ ID NO:2; and full-length residue 493 to residue 611 of SEQ ID NO:17). Within the metalloprotease domain of zsnk10 is a zinc-binding motif from residue 80 to residue 91 of SEQ ID NO:2 (also shown as residue 335 to residue 346 of SEQ ID NO:17). Within the disintegrin domain of zsnk10 is a disintegrin loop from residue 206 to residue 219 of SEQ ID NO:2 (also shown as from residue 461 to residue 474 of SEQ ID NO:17). The degenerate polynucleotide sequence for zsnk10 is shown in SEQ ID NOs: 3 and 18.

The polynucleotide sequences for zsnk11 are shown in SEQ ID NOs: 4 and 19. The polypeptide sequences for zsnk11 are shown in SEQ ID NOs:5 and 20. Domains of zsnkll include: signal domain (residue 1 to residue 18 of SEQID NO:20); a pro-peptide domain (residue 19 to residue 187 of SEQ ID NO:20); a metalloprotease domain (residue 1 to residue 226 as shown in SEQ ID NO:5, also shown as residue 191 to residue 393 of SEQ ID NO:20), and a disintegrin domain (residue 227 to residue 267 as shown in SEQ ID NO:5; also shown as residue 394 to residue 433 of SEQ ID NO:20). Within the metalloprotease domain of zsnkl 1 is a zinc-binding motif from residue 172 to residue 183 of SEQ ID NO:5 (also shown as residue 333 to residue 344 of SEQ ID NO:20). The degenerate polynucleotide sequence for zsnk11 is shown in SEQ ID NOs: 6 and 21.

The polynucleotide sequences for zsnk12 are shown in SEQ ID NOs: 7 and 22. The polypeptide sequences for zsnk12 are shown in SEQ ID NOs: 8 and 23. Domains of zsnk12 include: a secretion domain (residue 1 to residue 18 as shown in SEQ ID NO:23); a pro-peptide domain (residue 19 to residue 50 of SEQ ID NO:23); and a disintegrin-like domain (residue 71 to residue 134 as shown in SEQ ID NO:8, also shown as residue 51 to residue 110 of SEQ ID NO:23). Within the disintegrin domain of zsnk12 is a disintegrin loop from residue 107 to residue 119 of SEQ ID NO:8 (also shown as residue 85 to residue 97 of SEQ ID NO:23). The degenerate polynucleotide sequence for zsnk12 is shown in SEQ ID NO: 9.

Some members of the DP family have alternatively spliced isoforms. A protein which is an example of alternative splicing in the DPs is ADAM 12, also known as meltrin a. The truncated form of this molecule, which lacks the propeptide and metalloprotease domains, is associated with ectopic muscle formation in vivo, but not in vitro, indicating that cells expressing this gene produce a growth factor that acts on neighboring progenitor cells.

The present invention provides polynucleotide molecules, including DNA and RNA molecules, that encode the ZSNK10, ZSNK11, and ZSNK12 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the zsnk10 polypeptide of SEQ ID NO:2. SEQ ID NO:6 is a degenerate DNA sequence that encompasses all DNAs that encode the zsnk11 polypeptide of SEQ ID NO:5. SEQ ID NO:9 is a degenerate DNA sequence that encompasses all DNAs that encode the zsnk12 polypeptide of SEQ ID NO:8. SEQ ID NO: 18 is a degenerate DNA sequence that encompasses all DNAs that encode the zsnk10 polypeptide of SEQ ID NO:17. SEQ ID NO:21 is a degenerate DNA sequence that encompasses all DNAs that encode the zsnk11 polypeptide of SEQ ID NO:20. SEQ ID NO:24 is a degenerate DNA sequence that encompasses all DNAs that encode the zsnk12 polypeptide of SEQ ID NO:23. Those skilled in the art will recognize that the degenerate sequence of SEQ ID NOs:3, 6, 9, 18, 21, and 24 also provides all RNA sequences encoding SEQ ID NOs:2, 5, 8, 17, 20, and 23 by substituting U for T. Thus, zsnk10 polypeptide-encoding polynucleotides comprising nucleotide 1 to nucleotide 1200 of SEQ ID NO:3, and/or comprising nucleotide 1 to nucleotide 1833 of SEQ ID NO:18; zsnk11 polypeptide-encoding polynucleotides comprising nucleotide 1 to nucleotide 801 of SEQ ID NO:6, and/or comprising nucleotide 1 to nucleotide 1833 of SEQ ID NO:21; and zsnk12 polypeptide-encoding polynucleotides comprising nucleotide 1 to nucleotide 585 of SEQ ID NO:9, and/or comprising nucleotide 1 to nucleotide 333 of SEQ ID NO:24 , and their RNA equivalents are contemplated by the present invention. Table 1 sets forth the one-letter codes used within SEQ ID NOs:3, 6, 9, 18, 21, and 24 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOs:3, 6, 9, 18, 21, and 24, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gin | Q | CAA GAG | CAR |
| His | H | GAG CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAG TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NOs:2, 5, 8, 17, 20, and 23. Variant sequences can be readily tested for functionality as described herein.

One of ordinary skill in the art will also appreciate that different species can exhibit “preferential codon usage.” Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NOs:3, 6, 9, 18, 21, and 24 serve as templates for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NOs:1, 4, 7, 16, 19, and 22, or a sequence complementary thereto under stringent conditions. Polynucleotide hybridization is well known in the art and widely used for many applications, see for example, Sambrook et al., Molecular Cloning: A Laboratory Manual, Second Edition, Cold Spring Harbor, N.Y., 1989; Ausubel et al., eds., Current Protocols in Molecular Biology, John Wiley and Sons, Inc., NY, 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

As an illustration, a nucleic acid molecule encoding a variant ZSNK10, ZSNK11, or ZSNK12 polypeptide can be hybridized with a nucleic acid molecule having the nucleotide sequence of SEQ ID NOs:1, 4, 7, 16, 19, or 22 (or their complements) at 42° C. overnight in a solution comprising 50% formamide, 5×SSC (1×SSC: 0.15 M sodium chloride and 15 mM sodium citrate), 50 mM sodium phosphate (pH 7.6), 5×Denhardt's solution (100×Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g., ExpressHyb™ Hybridization Solution from CLONTECH Laboratories, Inc., Palo Alto, Calif.) according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant ZSNK10, ZSNK11, or ZSNK12 polypeptide hybridize with a nucleic acid molecule having the nucleotide sequences of SEQ ID NOs:1, 4, 7, 16, 19, or 22 (or their complements) under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

The present invention also contemplates ZSNK10, ZSNK11, or ZSNK12 variant nucleic acid molecules that can be identified using two criteria: a determination of the similarity between the encoded polypeptides with the amino acid sequences of SEQ ID NOs:2, 5, 8, 17, 20, and/or 23 (as described below), and a hybridization assay, as described above. Such ZSNK10, ZSNK11, or ZSNK12 variants include nucleic acid molecules (1) that hybridize with a nucleic acid molecule having the nucleotide sequence of SEQ ID NOs:1, 4, 7, 16, 19, or 22 (or their complements) under stringent washing conditions, in which the wash stringency is equivalent to 0.5×-2×SSC with 0.1% SDS at 55-65° C., and (2) that encode a polypeptide having at least 80%, preferably 90%, more preferably, 95% or greater than 95% sequence identity to the amino acid sequence of SEQ ID NOs:2, 5, 8, 17, 20, or 23. Alternatively, ZSNK10, ZSNK11, and ZSNK12 variants can be characterized as nucleic acid molecules (1) that hybridize with a nucleic acid molecule having the nucleotide sequence of SEQ ID NOs:1 or 3, 4 or 6, 7 or 9, 16 or 18, 19 or 21, or 22 or 24 (or their complements) under highly stringent washing conditions, in which the wash stringency is equivalent to 0.1×-0.2×SSC with 0.1% SDS at 50-65° C., and (2) that encode a polypeptide having at least 80%, preferably 90%, more preferably 95% or greater than 95% sequence identity to the amino acid sequence of SEQ ID NOs:2, 5, 8, 17, 20, or 23.

The highly conserved amino acids in the disintegrin domain of ZSNK10, ZSNK11, and ZSNK12 can be used as a tool to identify new family members. For instance, reverse transcription-polymerase chain reaction (RT-PCR) can be used to amplify sequences encoding the conserved disintegrin domain from RNA obtained from a variety of tissue sources or cell lines. In particular, highly degenerate primers designed from the ZSNK10, ZSNK11, and ZSNK12 sequences are useful for this purpose.

As previously noted, the isolated polynucleotides of the present invention include DNA and RNA. Methods for preparing DNA and RNA are well known in the art. In general, RNA is isolated from a tissue or cell that produces large amounts of ZSNK10, ZSNK11, and ZSNK12 RNA. Such tissues and cells can be identified by Northern blotting (Thomas, Proc. Natl. Acad. Sci. USA 77:5201, 1980), and include venom pouches of Sistrurus miliarius, and Agkistrodon piscivorus snakes.

Total RNA can be prepared using guanidine isothiocyante extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding ZSNK10, ZSNK11, and ZSNK12 polypeptides are then identified and isolated by, for example, hybridization or PCR.

A full-length clone encoding ZSNK10, ZSNK11, and ZSNK12 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to ZSNK10, ZSNK11, and ZSNK12 or other specific binding partners.

ZSNK10, ZSNK11, and ZSNK12 polynucleotide sequences disclosed herein can also be used as probes or primers to clone 5′ non-coding regions of a ZSNK10, ZSNK11, and ZSNK12 gene. Promoter elements from a ZSNK10, ZSNK11, and ZSNK12 gene could thus be used to direct the tissue-specific expression of heterologous genes in, for example, transgenic animals or patients treated with gene therapy. Cloning of 5′ flanking sequences also facilitates production of ZSNK10, ZSNK11, and ZSNK12 proteins by “gene activation” as disclosed in U.S. Pat. No. 5,641,670. Briefly, expression of an endogenous ZSNK10, ZSNK11, and ZSNK12 gene in a cell is altered by introducing into the ZSNK10, ZSNK11, and ZSNK12 locus a DNA construct comprising at least a targeting sequence, a regulatory sequence, an exon, and an unpaired splice donor site. The targeting sequence is a ZSNK10, ZSNK11, and ZSNK12 5′ non-coding sequence that permits homologous recombination of the construct with the endogenous ZSNK10, ZSNK11, and ZSNK12 locus, whereby the sequences within the construct become operably linked with the endogenous ZSNK10, ZSNK11, and ZSNK12 coding sequence. In this way, an endogenous ZSNK10, ZSNK11, and ZSNK12 promoter can be replaced or supplemented with other regulatory sequences to provide enhanced, tissue-specific, or otherwise regulated expression.

The polynucleotides of the present invention can also be synthesized using DNA synthesizers. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick and Pasternak, Molecular Biotechnology, Principles and Applications of Recombinant DNA, (ASM Press, Washington, D.C. 1994); Itakura et al., Annu. Rev. Biochem. 53: 323-356 (1984) and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are ZSNK10, ZSNK11, and ZSNK12 polypeptides from other mammalian species, including human, murine, porcine, ovine, bovine, canine, feline, equine, and other primate polypeptides. Orthologs of ZSNK10, ZSNK11, and ZSNK12 can be cloned using information and compositions provided by the present invention in combination with conventional cloning techniques. For example, a cDNA can be cloned using mRNA obtained from a tissue or cell type that expresses ZSNK10, ZSNK11, and ZSNK12 as disclosed herein. Such tissue would include, for example, venom pouches of poisonous snakes i.e., Sistrurus miliarius, and Agkistrodon piscivorus. Suitable sources of other mRNA can be identified by probing Northern blots with probes designed from the sequences disclosed herein. A library is then prepared from mRNA of a positive tissue or cell line. A human ZSNK10, ZSNK11, and ZSNK12-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human ZSNK10, ZSNK11, and ZSNK12 sequences disclosed herein. Within an additional method, the cDNA library can be used to transform or transfect host cells, and expression of the cDNA of interest can be detected with an antibody to ZSNK10, ZSNK11, and ZSNK12 polypeptide. Similar techniques can also be applied to the isolation of genomic clones.

Those skilled in the art will recognize that the sequences disclosed in SEQ ID NOs:1, 4, 7, 16, 19, and 22 represent a single allele of human ZSNK10, ZSNK11, and ZSNK12 and that allelic variation and alternative splicing are expected to occur. Allelic variants of this sequence can be cloned by probing cDNA or genomic libraries from different individuals according to standard procedures. Allelic variants of the DNA sequences shown in SEQ ID NOs:1, 4, 7, 16, 19, and 22, including those containing silent mutations and those in which mutations result in amino acid sequence changes, are within the scope of the present invention, as are proteins which are allelic variants of SEQ ID NOs:2, 5, 8, 17, 20, and 23. cDNAs generated from alternatively spliced mRNAs, which retain the properties of the ZSNK10, ZSNK11, and ZSNK12 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated ZSNK10, ZSNK11, and ZSNK12 polypeptides that are substantially similar to the polypeptides of SEQ ID NOs:2, 5, 8, 17, 20, and 23 and their orthologs. Such polypeptides will more preferably be at least 90% identical, and more preferably 95% or more identical to SEQ ID NOs:2, 5, 8, 17, 20, and 23 and their orthologs. The present invention also includes polypeptides that comprise an amino acid sequence having at at least 93%, preferably 95% or greater than 95% sequence identity to the disintegrin loop domains disclosed herein, i.e., residues 206 to 216 of SEQ ID NO:2 (or residue 461 to residue 474 of SEQ ID NO:17); and residues 106 to 119 of SEQ ID NO:8 (or residue 85 to residue 97 of SEQ NO:23). Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant ZSNK10, ZSNK11, and ZSNK12. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NOs:2, 5, 8, 17, 20, and 23) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from four to six.

The present invention includes nucleic acid molecules that encode a polypeptide having one or more conservative amino acid changes, compared with the amino acid sequences of SEQ ID NOs:2, 5, 8, 17, 20, and 23. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Conservative amino acid changes in an ZSNK10, ZSNK11, and ZSNK12 gene can be introduced by substituting nucleotides for the nucleotides recited in SEQ ID NOs:1, 4, 7, 16, 19, and 22. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to promote cell-cell interactions can be determined using a standard method, such as the assay described herein. Alternatively, a variant ZSNK10, ZSNK11, and ZSNK12 polypeptide can be identified by the ability to specifically bind anti-ZSNK10, ZSNK11, and ZSNK12 antibodies.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of disintegrin-integrin, or protease interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related disintegrin-like molecules.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed ZSNK10, ZSNK11, and ZSNK12 DNA and polypeptide sequences can be generated through DNA shuffling, as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., disintegrin-cell surface binding or protease activity) can be recovered from the host cells and rapidly sequenced using modem equipment. These methods allow the rapid determination of the importance of individual amino acid residues in a polypeptide of interest, and can be applied to polypeptides of unknown structure.

Regardless of the particular nucleotide sequence of a variant ZSNK10, ZSNK11, and ZSNK12 gene, the gene encodes a polypeptide that is characterized by its cell-cell interaction activity, or by the ability to bind specifically to an anti-ZSNK10, ZSNK11, and ZSNK12 antibody. More specifically, variant ZSNK10, ZSNK11, and ZSNK12 genes encode polypeptides which exhibit at least 50%, and preferably, greater than 70, 80, or 90%, of the activity of polypeptide encoded by the human ZSNK10, ZSNK11, and ZSNK12 gene described herein.

Variant ZSNK10, ZSNK11, and ZSNK12 polypeptides or substantially homologous ZSNK10, ZSNK11, and ZSNK12 polypeptides are characterized as having one or more amino acid substitutions, deletions or additions. These changes are preferably of a minor nature, that is conservative amino acid substitutions and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from 775 to 2000 amino acid residues that comprise a sequence that is at least 85%, preferably at least 90%, and more preferably 95% or more identical to the corresponding region of SEQ ID NOs:2, 5, 8, 17, 20, and 23. Polypeptides comprising affinity tags can further comprise a proteolytic cleavage site between the ZSNK10, ZSNK11, and ZSNK12 polypeptide and the affinity tag. Preferred such sites include thrombin cleavage sites and factor Xa cleavage sites.

For any ZSNK10, ZSNK11, and ZSNK12 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise ZSNK10, ZSNK11, and ZSNK12 variants based upon the nucleotide and amino acid sequences described herein. Accordingly, the present invention includes a computer-readable medium encoded with a data structure that provides at least one of the following sequences: SEQ ID NOs:1-24. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

The present invention further provides a variety of other polypeptide fusions and related multimeric proteins comprising one or more polypeptide fusions. For example, a disintegrin polypeptide domain can be prepared as a fusion to a dimerizing protein, as disclosed in U.S. Pat. Nos. 5,155,027 and 5,567,584. Preferred dimerizing proteins in this regard include other disintegrin polypeptide domains, disintegrin polypeptide domain fragments, or polypeptides comprising other members of the Disintegrin Protease family of proteins, such as, for example, members of the MDCs, SVMPs, and ADAMs. These disintegrin polypeptide domain fusions, disintegrin polypeptide domain fragment fusions, or fusions with other Disintegrin Proteases can be expressed in genetically engineered cells to produce a variety of multimeric disintegrin-like analogs.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a domain(s) conferring a biological function may be swapped between ZSNK10, ZSNK11, and ZSNK12 of the present invention with the functionally equivalent domain(s) from another family member, such as ADAM, MDC, and SVMP. Such domains include, but are not limited to, conserved motifs such as the secretory signal sequence, propeptide, protease, disintegrin and disintegrin loop domains, including the “RGD”, “DCD”, or “XXCD” (SEQ ID NO:14) sequence, the cysteine, transmembrane, and signalling domains. Such fusion proteins would be expected to have a biological functional profile that is the same or similar to polypeptides of the present invention or other known disintegrin-like family proteins (e.g. ADAMs, MDCs, and SVMPs), depending on the fusion constructed. Moreover, such fusion proteins may exhibit other properties as disclosed herein.

Moreover, using methods described in the art, polypeptide fusions, or hybrid ZSNK10, ZSNK11, and ZSNK12 proteins, are constructed using regions or domains of the inventive ZSNK10, ZSNK11, and ZSNK12 in combination with those of other disintegrin and disintegrin-like molecules. (e.g. ADAM, MDC, and SVMP), or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Auxiliary domains can be fused to ZSNK10, ZSNK11, and ZSNK12 polypeptides to target them to specific cells, tissues, or macromolecules (e.g., heart, peripheral blood, or brain). For example, a protease polypeptide domain, or protease polypeptide fragment or protein, could be targeted to a predetermined cell type by fusing it to a disintegrin polypeptide domain or fragment that specifically binds to an integrin polypeptide or integrin-like polypeptide on the surface of the target cell. In this way, polypeptides, polypeptide fragments and proteins can be targeted for therapeutic or diagnostic purposes. Such disintegrins or protease polypeptide domains or fragments can be fused to two or more moieties, such as an affinity tag for purification and a targeting-disintegrin domain. Polypeptide fusions can also comprise one or more cleavage sites, particularly between domains. See, Tuan et al., Connective Tissue Research 34:1-9, 1996.

Polypeptide fusions of the present invention will generally contain not more than about 1,500 amino acid residues, preferably not more than about 1,200 residues, more preferably not more than about 1,000 residues, and will in many cases be considerably smaller. For example, residues of ZSNK10, ZSNK11, and ZSNK12 polypeptide can be fused to E. coli β-galactosidase (1,021 residues; see Casadaban et al., J. Bacteriol. 143:971-980, 1980), a 10-residue spacer, and a 4-residue factor Xa cleavage site. In a second example, residues of ZSNK10, ZSNK11, and ZSNK12 polypeptide can be fused to maltose binding protein (approximately 370 residues), a 4-residue cleavage site, and a 6-residue polyhistidine tag.

To direct the export of a ZSNK10, ZSNK11, and ZSNK12 polypeptide from the host cell, the ZSNK10, ZSNK11, and ZSNK12 DNA is linked to a second DNA segment encoding a secretory peptide, such as a t-PA secretory peptide or a ZSNK10, ZSNK11, and ZSNK12 secretory peptide. To facilitate purification of the secreted polypeptide, a C-terminal extension, such as a poly-histidine tag, substance P, Flag peptide (Hopp et al., Bio/Technology 6:1204-1210, 1988; available from Eastman Kodak Co., New Haven, Conn.), maltose binding protein, or another polypeptide or protein for which an antibody or other specific binding agent is available, can be fused to the ZSNK10, ZSNK11, and ZSNK12 polypeptide.

The present invention also includes “functional fragments” of ZSNK10, ZSNK11, and ZSNK12 polypeptides and nucleic acid molecules encoding such functional fragments. Routine deletion analyses of nucleic acid molecules can be performed to obtain functional fragments of a nucleic acid molecule that encodes an ZSNK10, ZSNK11, and ZSNK12 polypeptide. As an illustration, DNA molecules having the nucleotide sequence of SEQ ID NOs:1, 4, 7, 16, 19, and 22 can be digested with Bal31 nuclease to obtain a series of nested deletions. The fragments are then inserted into expression vectors in proper reading frame, and the expressed polypeptides are isolated and tested for cell-cell interactions, or for the ability to bind anti-ZSNK10, ZSNK11, and ZSNK12 antibodies. One alternative to exonuclease digestion is to use oligonucleotide-directed mutagenesis to introduce deletions or stop codons to specify production of a desired fragment. Alternatively, particular fragments of an ZSNK10, ZSNK11, and ZSNK12 gene can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of a ZSNK10, ZSNK11, and ZSNK12 gene that have amino acid changes, compared with the amino acid sequence of SEQ ID NOs:2, 5, 8, 17, 20, and 23. A variant ZSNK10, ZSNK11, and ZSNK12 gene can be identified on the basis of structure by determining the level of identity with nucleotide and amino acid sequences of SEQ ID NOs:1, 2, 4, 5, 7, 8, 16, 17, 19, 20, 22, and 23 as discussed above. An alternative approach to identifying a variant gene on the basis of structure is to determine whether a nucleic acid molecule encoding a potential variant ZSNK10, ZSNK11, and ZSNK12 gene can hybridize to a nucleic acid molecule having the nucleotide sequence of SEQ ID NOs:1, 4, 7, 16, 19, and 22, as discussed above.

Using the methods discussed herein, one of ordinary skill in the art can identify and/or prepare a variety of polypeptide fragments or variants of SEQ ID NOs:2, 5, 8, 17, 20, and 23 or that retain the disintegrin and/or metalloprotease activity of the wild-type ZSNK10, ZSNK11, and ZSNK12 protein. Such polypeptides may include additional amino acids from, for example, a secretory domain, a propeptide domain, a protease domain, a disintegrin domain, a disintegrin loop (native or synthetic), part or all of a transmembrane and intracellular domains, including amino acids responsible for intracellular signaling; fusion domains; affinity tags; and the like.

The present invention also provides polypeptide fragments or peptides comprising an epitope-bearing portion of an ZSNK10, ZSNK11, and ZSNK12 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides contain at least four to ten amino acids, preferably at least ten to fifteen amino acids, more preferably 15 to 30 amino acids of SEQ ID NOs:2, 5, 8, 17, 20, and 23. Such epitope-bearing peptides and polypeptides can be produced by fragmenting a ZSNK10, ZSNK11, and ZSNK12 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Zsnk10, zsnk11, and zsnk12ing,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

As an illustration, potential antigenic sites in ZSNK10, ZSNK11, and ZSNK12 are identified using the Jameson-Wolf method, Jameson and Wolf, CABIOS 4:181, (1988), as implemented by the PROTEAN program (version 3.14) of LASERGENE (DNASTAR; Madison, Wis.). Default parameters are used in this analysis.

The results of this analysis of the polypeptide sequence of ZSNK10 indicated that a peptide consisting of amino acid residues 7 to 34 of SEQ ID NO:2; residues 26 to 33 of SEQ ID NO:2; residues 39 to 46 of SEQ ID NO:2; residues 66 to 71 of SEQ ID NO:2; residues 87 to 101 of SEQ ID NO:2; residues 106 to 134 of SEQ ID NO:2; residues 108 to 123 of SEQ ID NO:2; residues 127 to 134 of SEQ ID NO:2; residues 149 to 256 of SEQ ID NO:2; residues 160 to 167 of SEQ ID NO:2; residues 195 to 214 of SEQ ID NO:2; residues 222 to 246 of SEQ ID NO:2; residues 239 to 246 of SEQ ID NO:2; residues 248 to 256 of SEQ ID NO:2; residues 263 to 304 of SEQ ID NO:2; residues 270 to 280 of SEQ ID NO:2; residues 284 to 291 of SEQ ID NO:2; residues 294 to 302 of SEQ ID NO:2; residues 309 to 319 of SEQ ID NO:2; residues 323 to 329 of SEQ ID NO:2; residues 334 to 349 of SEQ ID NO:2; residues 375 to 384 of SEQ ID NO:2; residues 389 to 403 of SEQ ID NO:2, residues 157 to 163 of SEQ ID NO:17, residues 213 to 221 of SEQ ID NO:17, and residues 412 to 418 of SEQ ID NO:17. The results of this analysis of the polypeptide sequence of ZSNK11 indicated that a peptide consisting of amino acid residues 8 to 22 of SEQ ID NO:5; residues 9 to 18 of SEQ ID NO:5; residues 26 to 37 of SEQ ID NO:5; residues 28 to 35 of SEQ ID NO:5; residues 44 to 63 of SEQ ID NO:5; residues 52 to 58 of SEQ ID NO:5; residues 90 to 101 of SEQ ID NO:5; residues 112 to 125 of SEQ ID NO:5; residues 114 to 123 of SEQ ID NO:5; residues 147 to 163 of SEQ ID NO:5; residues 179 to 191 of SEQ ID NO:5; residues 181 to 186 of SEQ ID NO:5; residues 199 to 211 of SEQ ID NO:5; residues 216 to 226 of SEQ ID NO:5; residues 229 to 237 of SEQ ID NO:5; residues 246 to 267 of SEQ ID NO:5; residues 251 to 267 of SEQ ID NO:5, residues 155 to 162 of SEQ ID NO:20, residues 214 to 220 of SEQ ID NO:20, and residues 371 to 377 of SEQ ID NO:20. The results of this analysis of the polypeptide sequence of ZSNK12 indicated that a peptide consisting of amino acid residues 1 to 26 of SEQ ID NO:8; residues 18 to 15 of SEQ ID NO:8; residues 18 to 26 of SEQ ID NO:8; residues 46 to 81 of SEQ ID NO:8; residues 48 to 54 of SEQ HD NO:8; residues 58 to 68 of SEQ ID NO:8; residues 71 to 77 of SEQ ID NO:8; residues 83 to 132 of SEQ ID NO:8; residues 109 to 118 of SEQ ID NO:8; residues 120 to 132 of SEQ ID NO:8; residues 154 to 159 of SEQ ID NO:8; residues 165 to 175 of SEQ ID NO:8; residues 180 to 187 of SEQ ID NO:8, residues 82 to 90 of SEQ ID NO:23, and residues 102 to 109 of SEQ ID NO:23.

ZSNK10, ZSNK11, and ZSNK12 polypeptides can also be used to prepare antibodies that specifically bind to ZSNK10, ZSNK11, and ZSNK12 epitopes, peptides or polypeptides. The ZSNK10, ZSNK11, and ZSNK12 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a ZSNK10, ZSNK11, and ZSNK12 polypeptide (e.g., SEQ ID NOs:2, 5, 8, 17, 20, and 23). Polypeptides comprising a larger portion of a ZSNK10, ZSNK11, and ZSNK12 polypeptide, i.e., from 30 to 10 residues up to the entire length of the amino acid sequence are included. Antigens or immunogenic epitopes can also include attached tags, adjuvants and carriers, as described herein.

Suitable antigens also include the ZSNK10 polypeptides encoded by SEQ ID NO:2 from amino acid number 1 to amino acid number 356; the ZSNK11 polypeptides encoded by SEQ ID NO:5 from amino acid number 1 to amino acid number 267; and the ZSNK12 polypeptides encoded by SEQ ID NO:8 from amino acid number 1 to amino acid number 195 or a contiguous 9 to 860 amino acid fragment thereof. Other suitable antigens include ZSNK10 polypeptides from amino acid residue 1 to residue 140 of SEQ ID NO:2; residue 141 to residue 237 of SEQ ID NO:2; residue 238 to residue 356 of SEQ ID NO:2; residue 206 to residue 219 of SEQ ED NO:2; and residue 80 to 91 of SEQ ID NO:2. Other suitable antigens include ZSNK11 polypeptides from amino acid residue 1 to residue 226 of SEQ ID NO:5; residue 227 to residue 267 of SEQ ID NO:5; and residue 172 to 183 of SEQ ID NO:5. Other suitable antigens include ZSNK12 polypeptides from amino acid residue 1 to residue 25 of SEQ ID NO:8; residue 25 to residue 70 of SEQ ID NO:8; residue 71 to residue 134 of SEQ ID NO:8; and residue 106 to 119 of SEQ ID NO:8. Additional peptides to use as antigens are hydrophilic peptides such as those predicted by one of skill in the art from a hydrophobicity plot. Antibodies from an immune response generated by inoculation of an animal with these antigens can be isolated and purified as described herein. Methods for preparing and isolating polyclonal and monoclonal antibodies are well known in the art. See, for example, Current Protocols in Imunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a ZSNK10, ZSNK11, and ZSNK12 polypeptide or a fragment thereof. The immunogenicity of a ZSNK10, ZSNK11, and ZSNK12 polypeptide may be increased through the use of an adjuvant, such as alum (aluminum hydroxide) or Freund's complete or incomplete adjuvant. Polypeptides useful for immunization also include fusion polypeptides, such as fusions of ZSNK10, ZSNK11, and ZSNK12 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to ZSNK10, ZSNK11, and ZSNK12 protein or peptide, and selection of antibody display libraries in phage or similar vectors (for instance, through use of immobilized or labeled ZSNK10, ZSNK11, and ZSNK12 protein or peptide). Genes encoding polypeptides having potential ZSNK10, ZSNK11, and ZSNK12 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. NO. 5,571,698) and random peptide display libraries and kits for screening such libraries are available commercially, for instance from CLONTECH Laboratories, Inc., (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the ZSNK10, ZSNK11, and ZSNK12 sequences disclosed herein to identify proteins which bind to ZSNK10, ZSNK11, and ZSNK12. These “binding proteins” which interact with ZSNK10, ZSNK11, and ZSNK12 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as ZSNK10, ZSNK11, and ZSNK12 “antagonists” to block ZSNK10, ZSNK11, and ZSNK12 binding and signal transduction in vitro and in vivo. These anti-ZSNK10, ZSNK11, and ZSNK12 binding proteins would be useful for modulating, for example, platelet aggregation, apoptosis, neurogenesis, myogenesis, immunologic recognition, tumor formation, and cell-cell interactions in general.

Antibodies are determined to be specifically binding if they exhibit a threshold level of binding activity (to a ZSNK10, ZSNK11, and ZSNK12 polypeptide, peptide or epitope) of at least 10-fold greater than the binding affinity to a control (non-ZSNK10, ZSNK11, and ZSNK12) polypeptide. The binding affinity of an antibody can be readily determined by one of ordinary skill in the art, for example, by Scatchard analysis (Scatchard, G., Ann. NY Acad. Sci. 51: 660-672, 1949).

A variety of assays known to those skilled in the art can be utilized to detect antibodies which specifically bind to ZSNK10, ZSNK11, and ZSNK12 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant ZSNK10, ZSNK11, and ZSNK12 protein or polypeptide.

Antibodies to ZSNK10, ZSNK11, and ZSNK12 may be used for tagging cells that express ZSNK10, ZSNK11, and ZSNK12; for isolating ZSNK10, ZSNK11, and ZSNK12 by affinity purification; for diagnostic assays for determining circulating levels of ZSNK10, ZSNK11, and ZSNK12 polypeptides; for detecting or quantitating soluble ZSNK10, ZSNK11, and ZSNK12 as marker of underlying pathology or disease; in analytical methods employing FACS; for screening expression libraries; for generating anti-idiotypic antibodies; and as neutralizing antibodies or as antagonists to block ZSNK10, ZSNK11, and ZSNK12 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to ZSNK10, ZSNK11, and ZSNK12 or fragments thereof may be used in vitro to detect denatured ZSNK10, ZSNK11, and ZSNK12 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (integrin or antigen, respectively, for instance). More specifically, ZSNK10, ZSNK11, and ZSNK12 polypeptides or anti-ZSNK10, ZSNK11, and ZSNK12 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/ anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, a fusion protein including only the disintegrin domain may be suitable for directing a detectable molecule, a cytotoxic molecule or a complementary molecule to a cell or tissue type of interest. Similarly, the corresponding integrin to ZSNK10, ZSNK11, and ZSNK12 can be conjugated to a detectable or cytotoxic molecule and provide a generic targeting vehicle for cell/tissue-specific delivery of generic anti-complementary-detectable/cytotoxic molecule conjugates.

In another embodiment, ZSNK10, ZSNK11, and ZSNK12-cytokine fusion proteins or antibody-cytokine fusion proteins can be used for enhancing in vivo killing of target tissues if the ZSNK10, ZSNK11, and ZSNK12 polypeptide or anti-ZSNK10, ZSNK11, and ZSNK12 antibody targets such hyperproliferative tissues. (See, generally, Hornick et al., Blood 89:4437-47, 1997). They described fusion proteins that enable targeting of a cytokine to a desired site of action, thereby providing an elevated local concentration of cytokine. Suitable ZSNK10, ZSNK11, and ZSNK12 polypeptides or anti-ZSNK10, ZSNK11, and ZSNK12 antibodies target an undesirable cell or tissue (i.e., a tumor or a leukemia), and the fused cytokine mediates improved target cell lysis by effector cells. Suitable cytokines for this purpose include interleukin 2 and granulocyte-macrophage colony-stimulating factor (GM-CSF), for instance.

In yet another embodiment, if the ZSNK10, ZSNK11, and ZSNK12 polypeptide or anti-ZSNK10, ZSNK11, and ZSNK12 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis are significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

The ZSNK10, ZSNK11, and ZSNK12 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a ZSNK10, ZSNK11, and ZSNK12 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZSNK10, ZSNK11, and ZSNK12 polypeptide into the secretory pathway of a host cell, a secretory signal sequence (also known as a leader sequence, prepro sequence or pre sequence) is provided in the expression vector. The secretory signal sequence may be that of ZSNK10, ZSNK11, and ZSNK12, or may be derived from another secreted protein (e.g., t-PA) or synthesized de novo. The secretory signal sequence is operably linked to the ZSNK10, ZSNK11, and ZSNK12 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

The native secretory signal sequence of the polypeptides of the present invention is used to direct other polypeptides into the secretory pathway. The present invention provides for such fusion polypeptides. A signal fusion polypeptide can be made wherein a secretory signal sequence derived from a ZSNK10, ZSNK11, and ZSNK12 polypeptide is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein, such as a receptor. Such fusions may be used in vivo or in vitro to direct peptides through the secretory pathway.

Alternatively, the protease domain of ZSNK10, ZSNK11, and ZSNK12 can be substituted by a heterologous sequence providing a different protease domain. In this case, the fusion product can be secreted, and the disintegrin domain of ZSNK10, ZSNK11, and ZSNK12 can direct the protease domain to a specific tissue described above. This substituted protease domain can be chosen from the protease domains represented by the DP protein families, or domains from other known proteases.

Similarly, the disintegrin domain of ZSNK10, ZSNK11, and ZSNK12 protein can be substituted by a heterlogous sequence providing a different disintegrin domain. Again, the fusion product can be secreted and the substituted disintegrin domain can target the protease domain of ZSNK10, ZSNK11, and ZSNK12 to a specific tissue. The substituted disintegrin domain can be chosen from the disintegrin domains of the DP protein families. In these cases, the fusion products can be soluble or membrane-anchored proteins.

Cultured mammalian cells are suitable hosts within the present invention. Methods for introducing exogenous DNA into mammalian host cells include calcium phosphate-mediated transfection (Wigler et al., Cell 114:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins, such as CD4, CD8, Class I MHC, or placental alkaline phosphatase, may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant ZSNK10, ZSNK11, and ZSNK12 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBacl™ (Life Technologies) containing a Tn7 transposon to move the DNA encoding the ZSNK10, ZSNK11, and ZSNK12 polypeptide into a baculovirus genome maintained in E. coli as a large plasmid called a “bacmid.” The pFastBacl™ transfer vector utilizes the AcNPV polyhedrin promoter to drive the expression of the gene of interest, in this case ZSNK10, ZSNK11, and ZSNK12. However, pFastBacl™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol Chem 270:1543-9, 1995. In such transfer vector constructs, a short or long version of the basic protein promoter can be used. Moreover, transfer vectors can be constructed which replace the native ZSNK10, ZSNK11, and ZSNK12 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native ZSNK10, ZSNK11, and ZSNK12 secretory signal sequence. In addition, transfer vectors can include an in-frame fusion with DNA encoding an epitope tag at the C- or N-terminus of the expressed ZSNK10, ZSNK11, and ZSNK12 polypeptide, for example, a Glu-Glu epitope tag (Grussenmeyer, T. et al., Proc. Natl. Acad. Sci. 82:7952-4, 1985). Using a technique known in the art, a transfer vector containing ZSNK10, ZSNK11, and ZSNK12 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses ZSNK10, ZSNK11, and ZSNK12 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cell405™ (JRH Biosciences, Lenexa, Ks.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the ZSNK10, ZSNK11, and ZSNK12 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,716,808, 5,736,383, 5 5,854,039, and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a ZSNK10, ZSNK11, and ZSNK12 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:2145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification.

Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for ZSNK10, ZSNK11, and ZSNK12 amino acid residues.

It is preferred to purify the polypeptides of the present invention to >80% purity, more preferably to >90% purity, even more preferably >95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant ZSNK10, ZSNK11, and ZSNK12 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., ibid. Maltose binding protein fusions are purified on an amylose column according to methods known in the art.

The polypeptides of the present invention can be isolated by a combination of procedures including, but not limited to, anion and cation exchange chromatography, size exclusion, and affinity chromatography. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, an immunoglobulin domain) may be constructed to facilitate purification.

ZSNK10, ZSNK11, and ZSNK12 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, ZSNK10, ZSNK11, and ZSNK12 proteins can be prepared as monomers or multimers; glycosylated or non-glycosylated; pegylated or non-pegylated; and may or may not include an initial methionine amino acid residue.

The disintegrin loop of ZSNK10 (i.e., from residue 206 to residue 219 of SEQ ID NO:2, from residue 461 to residue 474 of SEQ ID NO:17) are of particular interest for use in assays and treatment of disorders of the heart and brain. This peptide can be synthesized as a linear peptide or a disulfide linked peptide. Peptides having disulfide bonds, for example, between residues 206 and 213 of SEQ ID NO:2, between residues 213 and 219 of SEQ ID NO:2, between residues 461 and 468 of SEQ ID NO:17, or between residues 468 and 474 of SEQ ID NO:23, are also of interest. Similarly the disintegrin loop of ZSNK12 (i.e., from residue 107 to residue 119 of SEQ ID NO:8, or from residue 85 to residue 97 of SEQ ID NO:23) with disulfide linkages between residues 107 and 119 of SEQ ID NO:8, or between residues 85 and 97 of SEQ ID NO:23, are also of interest. See Jia, L. G., ibid for additional description of peptide synthesis and disulfide linkages.

The activity of ZSNK10, ZSNK11, and ZSNK12 polypeptides can be measured using a variety of assays that measure, for example, cell-cell interactions; proteolysis; extracellular matrix formation or remodeling; metastasis, and other biological functions associated with disintegrin family members or with integrin/disintegrin interactions, such as, apoptosis; or differentiation, for example. Of particular interest is a change in platelet aggregation. Assays measuring platelet aggregation are well known in the art. For a general reference, see Dennis, PNAS 87: 2471-2475, 1989.

Proteins, including alternatively spliced peptides, of the present invention are useful for tumor suppression, and growth and differentiation either working in isolation, or in conjunction with other molecules (growth factors, cytokines, etc.). Alternative splicing of ZSNK10, ZSNK11, and ZSNK12 may cell-type specific and confer activity to specific tissues.

Another assay of interest measures or detects changes in proliferation, differentiation, development and/or and electrical coupling of muscle cells or myocytes. Additionally, the effects of a ZSNK10, ZSNK11, and ZSNK12 polypeptides on cell-cell interactions of fibroblasts, myoblasts, nerve cells, white blood cells, immune cells, gamete cells or cells, in general, of a reproductive nature, and tumor cells would be of interest to measure. Yet other assays examines changes in protease activity and apoptosis.

The activity of molecules of the present invention can be measured using a variety of assays that, for example, measure neogenesis or hyperplasia (i.e., proliferation) of cardiac cells based on the tissue specificity in adult heart. Additional activities likely associated with the polypeptides of the present invention include proliferation of endothelial cells, cardiomyocytes, fibroblasts, skeletal myocytes directly or indirectly through other growth factors; action as a chemotaxic factor for endothelial cells, fibroblasts and/or phagocytic cells; osteogenic factor; and factor for expanding mesenchymal stem cell and precursor populations.

Proliferation can be measured using cultured cardiac cells or in vivo by administering molecules of the claimed invention to an appropriate animal model. Generally, proliferative effects are observed as an increase in cell number and therefore, may include inhibition of apoptosis, as well as mitogenesis. Cultured cells include cardiac fibroblasts, cardiac myocytes, skeletal myocytes, human umbilical vein endothelial cells from primary cultures. Established cell lines include: NIH 3T3 fibroblast (ATCC No. CRL-1658), CHH-1 chum heart cells (ATCC No. CRL-1680), H9c2 rat heart myoblasts (ATCC No. CRL-1446), Shionogi mammary carcinoma cells (Tanaka et al., Proc. Natl. Acad. Sci. 89:8928-8932, 1992) and LNCap.FGC adenocarcinoma cells (ATCC No. CRL-1740). Assays measuring cell proliferation are well known in the art. For example, assays measuring proliferation include such assays as chemosensitivity to neutral red dye (Cavanaugh et al., Investigational New Drugs 8:347-354, 1990), incorporation of radiolabelled nucleotides (Cook et al., Analytical Biochem. 179:1-7, 1989), incorporation of 5-bromo-2′-deoxyuridine (BrdU) in the DNA of proliferating cells (Porstmann et al., J. Immunol. Methods 82:169-179, 1985), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988).

To determine if ZSNK10, ZSNK11, and ZSNK12 is a chemotractant in vivo, ZSNK10, ZSNK11, and ZSNK12 can be given by intradermal or intraperitoneal injection. Characterization of the accumulated leukocytes at the site of injection can be determined using lineage specific cell surface markers and fluorescence immunocytometry or by immunohistochemistry (Jose, J. Exp. Med. 179:881-87, 1994). Release of specific leukocyte cell populations from bone marrow into peripheral blood can also be measured after ZSNK10, ZSNK11, and ZSNK12 injection.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation.

Differentiation markers that are expressed exclusively by mature cells are usually functional properties such as cell products, enzymes to produce cell products and receptors and receptor-like complementary molecules. The stage of a cell population's differentiation is monitored by identification of markers present in the cell population. For example, myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The existence of early stage cardiac myocyte progenitor cells (often referred to as cardiac myocyte stem cells) has been speculated, but not demonstrated, in adult cardiac tissue. The novel polypeptides of the present invention are useful for studies to isolate mesenchymal stem cells and cardiac myocyte progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, ZSNK10, ZSNK11, and ZSNK12 polypeptides, or their orthologs, may stimulate inhibition or proliferation of endocrine and exocrine cells.

Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989).0

The ZSNK10, ZSNK11, and ZSNK12 polypeptides of the present invention can be used to study proliferation or differentiation in human tissues. Such methods of the present invention generally comprise incubating cells derived from these tissues in the presence and absence of ZSNK10, ZSNK11, and ZSNK12 polypeptide, monoclonal antibody, agonist or antagonist thereof and observing changes in cell proliferation or differentiation. Cell lines from these tissues are commercially available from, for example, American Type Culture Collection (Manasas, Va.).

Proteins, including alternatively spliced peptides, and fragments, of the present invention are useful for studying cell-cell interactions, fertilization, development, immune recognition, growth control, and tumor suppression. ZSNK10, ZSNK11, and ZSNK12 molecules, variants, and fragments can be applied in isolation, or in conjunction with other molecules (growth factors, cytokines, etc).

Proteins of the present invention are useful for delivery of therapeutic agents such as, but not limited to, proteases, radionuclides, chemotherapy agents, and small molecules. Effects of these therapeutic agents can be measured in vitro using cultured cells, ex vivo on tissue slices, or in vivo by administering molecules of the claimed invention to the appropriate animal model. An alternative in vivo approach for assaying proteins of the present invention involves viral delivery systems. Exemplary viruses for this purpose include adenovirus, herpesvirus, lentivirus, vaccinia virus and adeno-associated virus (AAV). Adenovirus, a double-stranded DNA virus, is currently the best studied gene transfer vector for delivery of heterologous nucleic acid (for a review, see T. C. Becker et al., Meth. Cell Biol. 43:161-89, 1994; and J. T. Douglas and D. T. Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Gamier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

As a soluble or cell-surface protein, the activity of ZSNK10, ZSNK11, and ZSNK12 polypeptide or a peptide to which ZSNK10, ZSNK11, and ZSNK12 binds, can be measured by a silicon-based biosensor microphysiometer which measures the extracellular acidification rate or proton excretion associated with cell-surface protein interactions and subsequent physiologic cellular responses. An exemplary device is the CytosensorT Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including ZSNK10, ZSNK11, and ZSNK12 proteins, their, agonists, and antagonists. Preferably, the microphysiometer is used to measure responses of a ZSNK10, ZSNK11, and ZSNK12-responsive eukaryotic cell, compared to a control eukaryotic cell that does not respond to ZSNK10, ZSNK11, and ZSNK12 polypeptide. ZSNK10, ZSNK11, and ZSNK12-responsive eukaryotic cells comprise cells into which a polynucleotide for ZSNK10, ZSNK11, and ZSNK12 has been transfected creating a cell that is responsive to ZSNK10, ZSNK11, and ZSNK12; or cells naturally responsive to ZSNK10, ZSNK11, and ZSNK12. Differences, measured by a change in the response of cells exposed to ZSNK10, ZSNK11, and ZSNK12 polypeptide, relative to a control not exposed to ZSNK10, ZSNK11, and ZSNK12, are a direct measurement of ZSNK10, ZSNK11, and ZSNK12-modulated cellular responses. Moreover, such ZSNK10, ZSNK11, and ZSNK12-modulated responses can be assayed under a variety of stimuli. The present invention provides a method of identifying agonists and antagonists of ZSNK10, ZSNK11, and ZSNK12 protein, comprising providing cells responsive to a ZSNK10, ZSNK11, and ZSNK12 polypeptide, culturing a first portion of the cells in the absence of a test compound, culturing a second portion of the cells in the presence of a test compound, and detecting a change in a cellular response of the second portion of the cells as compared to the first portion of the cells. The change in cellular response is shown as a measurable change in extracellular acidification rate. Moreover, culturing a third portion of the cells in the presence of ZSNK10, ZSNK11, and ZSNK12 polypeptide and the absence of a test compound provides a positive control for the ZSNK10, ZSNK11, and ZSNK12-responsive cells, and a control to compare the agonist activity of a test compound with that of the ZSNK10, ZSNK11, and ZSNK12 polypeptide. Antagonists of ZSNK10, ZSNK11, and ZSNK12 can be identified by exposing the cells to ZSNK10, ZSNK11, and ZSNK12 protein in the presence and absence of the test compound, whereby a reduction in ZSNK10, ZSNK11, and ZSNK12-stimulated activity is indicative of agonist activity in the test compound.

Moreover, ZSNK10, ZSNK11, and ZSNK12 can be used to identify cells, tissues, or cell lines which respond to a ZSNK10, ZSNK11, and ZSNK12-stimulated pathway. The microphysiometer, described above, can be used to rapidly identify disintegrin-responsive cells, such as cells responsive to ZSNK10, ZSNK11, and ZSNK12 of the present invention. Cells can be cultured in the presence or absence of ZSNK10, ZSNK11, and ZSNK12 polypeptide. Those cells which elicit a measurable change in extracellular acidification in the presence of ZSNK10, ZSNK11, and ZSNK12 are responsive to ZSNK10, ZSNK11, and ZSNK12. Such cell lines, can be used to identify integrins, antagonists and agonists of ZSNK10, ZSNK11, and ZSNK12 polypeptide as described above. Using similar methods, cells expressing ZSNK10, ZSNK11, and ZSNK12 can be used to identify cells which stimulate a ZSNK10, ZSNK11, and ZSNK12-signalling pathway.

ZSNK10, ZSNK11, and ZSNK12 peptides, agonists (including the native disintegrin and protease domains, as well as a native or synthetic disintegrin loop peptide) and antagonists have enormous potential in both in vitro and in vivo applications. Compounds identified as ZSNK10, ZSNK11, and ZSNK12 agonists and antagonists are useful for studying cell-cell interactions, myogenesis, apoptosis, neurogenesis, tumor proliferation and suppression, extracellular matrix proteins, repair and remodeling of ischemia reperfusion and inflammation in vitro and in vivo. For example, ZSNK10, ZSNK11, and ZSNK12 and agonist compounds are useful as components of defined cell culture media, and may be used alone or in combination with other cytokines and hormones to replace serum that is commonly used in cell culture. Agonists are thus useful in specifically promoting the growth and/or development of cells of the myeloid and lymphoid lineages in culture. Additionally, ZSNK10, ZSNK11, and ZSNK12 polypeptides and ZSNK10, ZSNK11, and ZSNK12 agonists, including small molecules are useful as a research reagent, such as for the expansion, differentiation, and/or cell-cell interactions of tissues. ZSNK10, ZSNK11, and ZSNK12 polypeptides are added to tissue culture media.

Antagonists are also useful as research reagents for characterizing sites of interactions between members of complement/anti-complement pairs as well as sites of cell-cell interactions. Inhibitors of ZSNK10, ZSNK11, and ZSNK12 activity (ZSNK10, ZSNK11, and ZSNK12 antagonists) include anti-ZSNK10, anti-ZSNK11, and anti-ZSNK12 antibodies aw well as soluble ZSNK10, ZSNK11, and ZSNK12 polypeptides (such as in SEQ ID NOs:2, 5, 8, 17, 20, and 23), as well as other peptidic and non-peptidic agents (including ribozymes).

ZSNK10, ZSNK11, and ZSNK12 can also be used to identify inhibitors (antagonists) of its activity. Test compounds are added to the assays disclosed herein to identify compounds that inhibit the activity of ZSNK10, ZSNK11, and ZSNK12. In addition to those assays disclosed herein, samples can be tested for inhibition of ZSNK10, ZSNK11, and ZSNK12 activity within a variety of assays designed to measure disintegrin/integrin binding or the stimulation/inhibition of ZSNK10, ZSNK11, and ZSNK12-dependent cellular responses. For example, ZSNK10, ZSNK11, and ZSNK12-responsive cell lines can be transfected with a reporter gene construct that is responsive to a ZSNK10, ZSNK11, and ZSNK12-stimulated cellular pathway. Reporter gene constructs of this type are known in the art, and will generally comprise a DNA response element operably linked to a gene encoding an assayable protein, such as luciferase, or a metabolite, such as cyclic AMP. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):287-94; 1990. Hormone response elements are reviewed in Beato, Cell 56:335-44; 1989. The most likely reporter gene construct would contain a disintegrin that, upon binding an integrin, would signal intracellularly through, for example, a SRE reporter. Candidate compounds, solutions, mixtures or extracts are tested for the ability to inhibit the activity of ZSNK10, ZSNK11, and ZSNK12 on the target cells, as evidenced by a decrease in ZSNK10, ZSNK11, and ZSNK12 stimulation of reporter gene expression. Assays of this type will detect compounds that directly block ZSNK10, ZSNK11, and ZSNK12 binding to a cell-surface protein, i.e., integrin, or the anti-complementary member of a complementary/anti-complementary pair, as well as compounds that block processes in the cellular pathway subsequent to complement/anti-complement binding. In the alternative, compounds or other samples can be tested for direct blocking of ZSNK10, ZSNK11, and ZSNK12 binding to a integrin using ZSNK10, ZSNK11, and ZSNK12 tagged with a detectable label (e.g., ¹²⁵I, biotin, horseradish peroxidase, FITC, and the like). Within assays of this type, the ability of a test sample to inhibit the binding of labeled ZSNK10, ZSNK11, and ZSNK12 to the integrin is indicative of inhibitory activity, which can be confirmed through secondary assays. Integrins used within binding assays may be cellular integrins, soluble integrins, or isolated, immobilized integrins.

The amino acid sequence comprising the “RGD” integrin binding component of ZSNK12, (i.e., residues 107 to 119 of SEQ ID NO: 8, or residues 85 to 97 of SEQ ID NO:23) may also be used as an inhibitor. Similarly the amino acid sequence comprising the “DDCD” sequence (SEQ ID NO:15) of ZSNK10 (i.e., residues 206 to 219 of SEQ ID NO:2, or residues 461 to 474 of SEQ ID NO:17) which is analogous to the “RGD” integrin binding loop, may also be used as an inhibitor. Such an inhibitor would bind an integrin other than its naturally occurring integrin by nature of its folding structure. Particular interests in such an inhibitor would be to mediate platelet aggregation, gamete maturation, or immunologic response. Assays measuring binding and inhibition are known in the art.

A ZSNK10, ZSNK11, and ZSNK12 ligand-binding polypeptide can also be used for purification of ligand. The polypeptide is immobilized on a solid support, such as beads of agarose, cross-linked agarose, glass, cellulosic resins, silica-based resins, polystyrene, cross-inked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing integrins are passed through the column one or more times to allow integrins to bind to the integrin binding loop polypeptide. The integrin is then eluted using changes in salt concentration, chaotropic agents (guanidine HCl), or pH to disrupt integrin, or receptor binding.

An assay system that uses a ligand-binding receptor (or an antibody, one member of a complementary/anti-complementary pairor other cell-surface binding protein) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member, disintegrin or fragment is covalently attached, using amine or sulfhydryl chemistry, to dextran fibers that are attached to gold film within the flow cell. A test sample is passed through the cell. If an integrin, epitope, or opposite member of the complementary/anti-complementary pair is present in the sample, it will bind to the immobilized disintegrin, antibody or member, respectively, causing a change in the refractive index of the medium, which is detected as a change in surface plasmon resonance of the gold film. This system allows the determination of on- and off-rates, from which binding affinity can be calculated, and assessment of.

Integrin polypeptides and other receptor polypeptides which bind disintegrin polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

A “soluble protein” is a protein that is not bound to a cell membrane. Soluble proteins are most commonly ligand-binding receptor polypeptides that lack transmembrane and cytoplasmic domains. Soluble proteins can comprise additional amino acid residues, such as affinity tags that provide for purification of the polypeptide or provide sites for attachment of the polypeptide to a substrate, or immunoglobulin constant region sequences. Many cell-surface proteins have naturally occurring, soluble counterparts that are produced by proteolysis or translated from alternatively spliced mRNAs. Proteins are said to be substantially free of transmembrane and intracellular polypeptide segments when they lack sufficient portions of these segments to provide membrane anchoring or signal transduction, respectively.

Molecules of the present invention can be used to identify and isolate integrins, or members of complement/anti-complement pairs involved in cell-cell interactions. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified.

The molecules of the present invention will be useful in repair and remodeling after an ischemic event, modulating immunologic recognition, and/or platelet aggregation. The polypeptides, nucleic acid and/or antibodies of the present invention can be used in treatment of disorders associated with infarct in brain or heart tissue, and/or platelet aggregation. The molecules of the present invention can be used to modulate proteolysis, apoptosis, neurogenesis, myogenesis, cell adhesion, cell fusion, and signaling or to treat or prevent development of pathological conditions in diverse tissue, including heart, peripheral blood, and brain. In particular, certain diseases may be amenable to such diagnosis, treatment or prevention. The molecules of the present invention can be used to modulate inhibition and proliferation of neurons and myocytes in these and other tissues. Disorders which may be amenable to diagnosis, treatment or prevention with ZSNK10, ZSNK11, and ZSNK12 polypeptides, their agonists or antagonists include, for example, Alzheimers's Disease, tumor formation, Multiple Sclerosis, Congestive Heart Failure, Ischemic Reperfusion or infarct, coagulation disorders, and degenerative diseases.

Additionally, the propeptide domain, comprising residues 31 to 200, can be used as a modulator of protease activity of other DP family members as well as other proteases, in general. Polypeptides and polynucleotides encoding them can be used as a soluble molecule or as a fusion product to regulate such proteases.

Polynucleotides encoding ZSNK10, ZSNK11, and ZSNK12 polypeptides are useful within gene therapy applications where it is desired to increase or inhibit ZSNK10, ZSNK11, and ZSNK12 activity. If a mammal has a mutated or absent ZSNK10, ZSNK11, and ZSNK12 gene, the ZSNK10, ZSNK11, and ZSNK12 gene can be introduced into the cells of the mammal. In one embodiment, a gene encoding a ZSNK10, ZSNK11, and ZSNK12 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a ZSNK10, ZSNK11, and ZSNK12 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Similarly, the ZSNK10, ZSNK11, and ZSNK12 polynucleotides (SEQ ID NOs:1, 4, 7, 16, 19, or 22) can be used to target specific tissues. It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Various techniques, including antisense and ribozyme methodologies, can be used to inhibit ZSNK10, ZSNK11, and ZSNK12 gene transcription and translation, such as to inhibit cell proliferation in vivo. Polynucleotides that are complementary to a segment of a ZSNK10, ZSNK11, and ZSNK12-encoding polynucleotide (e.g., a polynucleotide as set forth in SEQ ID NOs:1, 4, 7, 16, 19, and 22, or their degenerate sequences) are designed to bind to ZSNK10, ZSNK11, and ZSNK12-encoding mRNA and to inhibit translation of such mRNA. Such antisense polynucleotides are used to inhibit expression of ZSNK10, ZSNK11, and ZSNK12 polypeptide-encoding genes in cell culture or in a subject.

Mice engineered to express the ZSNK10, ZSNK11, and ZSNK12 gene, referred to as “transgenic mice,” and mice that exhibit a complete absence of ZSNK10, ZSNK11, and ZSNK12 gene function, referred to as “knockout mice,” may also be generated (Snouwaert et al., Science 257:283, 1992), may also be generated (Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express ZSNK10, ZSNK11, and ZSNK12, either ubiquitously or under a tissue-specific or tissue-restricted promoter can be used to ask whether over-expression causes a phenotype. For example, over-expression of a wild-type ZSNK10, ZSNK11, and ZSNK12 polypeptide, polypeptide fragment or a mutant thereof may alter normal cellular processes, resulting in a phenotype that identifies a tissue in which ZSNK10, ZSNK11, and ZSNK12 expression is functionally relevant and may indicate a therapeutic target for the ZSNK10, ZSNK11, and ZSNK12, its agonists or antagonists. For example, a preferred transgenic mouse to engineer is one that over-expresses the soluble ZSNK10, ZSNK11, and ZSNK12 polypeptide (approximately amino acids 28 to 802 of SEQ ID NO:2, amino acids 19 to 611 of SEQ ID NO:17, amino acids 19 to 433 of SEQ ID NO:20, amino acids 19 to 110 if SEQ ID NO:23). Moreover, such over-expression may result in a phenotype that shows similarity with human diseases. Similarly, knockout ZSNK10, ZSNK11, and ZSNK12 mice can be used to determine where ZSNK10, ZSNK11, and ZSNK12 is absolutely required in vivo. The phenotype of knockout mice is predictive of the in vivo effects of that a ZSNK10, ZSNK11, and ZSNK12 antagonist, such as those described herein, may have. The human ZSNK10, ZSNK11, and ZSNK12 cDNA can be used to isolate murine ZSNK10, ZSNK11, and ZSNK12 mRNA, cDNA and genomic DNA, which are subsequently used to generate knockout mice. These mice may be employed to study the ZSNK10, ZSNK11, and ZSNK12 gene and the protein encoded thereby in an in vivo system, and can be used as in vivo models for corresponding human diseases. Moreover, transgenic mice expression of ZSNK10, ZSNK11, and ZSNK12 antisense polynucleotides or ribozymes directed against ZSNK10, ZSNK11, and ZSNK12, described herein, can be used analogously to transgenic mice described above.

ZSNK10, ZSNK11, and ZSNK12 polypeptides, variants, and fragments thereof, may be useful as replacement therapy for disorders associated with cell-cell interactions, including disorders related to, for example, fertility, gamete maturation, immunology, coagulation, trauma, and epithelial disorders, in general.

A less widely appreciated determinant of tissue morphogenesis is the process of cell rearrangement: Both cell motility and cell-cell adhesion are likely to play central roles in morphogenetic cell rearrangements. Cells need to be able to rapidly break and probably simultaneously remake contacts with neighboring cells. See Gumbiner, B. M., Cell 69:385-387, 1992. As a secreted protein, ZSNK10, ZSNK11, and ZSNK12 can also play a role in intercellular rearrangement in tissues.

The human orthologs of ZSNK10, ZSNK11, and ZSNK12 genes may be useful to as a probe to identify humans who have a defective ZSNK10, ZSNK11, and ZSNK12 gene. Thus, polynucleotides and polypeptides of ZSNK10, ZSNK11, and ZSNK12, their orthologs, and mutations to them, can be used a diagnostic indicators of cancer in human tissues.

The polypeptides of the present invention are useful in studying cell adhesion and the role thereof in metastasis and may be useful in preventing metastasis. Similarly, polynucleotides and polypeptides of ZSNK10, ZSNK11, and ZSNK12 may be used to replace their defective counterparts in tumor or malignant tissues.

The polynucleotides of the present invention may also be used in conjunction with a regulatable promoter, thus allowing the dosage of delivered protein to be regulated.

The chromosomal localization of the human orthologs of ZSNK10, ZSNK11, and ZSNK12 can be determined. Thus, the present invention also provides reagents which will find use in diagnostic applications. For example, the ZSNK10, ZSNK11, and ZSNK12 gene, a probe comprising ZSNK10, ZSNK11, and ZSNK12 DNA or RNA or a subsequence thereof can be used to determine if the human ortholog of ZSNK10, ZSNK11, and ZSNK12 gene is present or if a mutation has occurred. Detectable chromosomal aberrations at the ZSNK10, ZSNK11, and ZSNK12 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

For pharmaceutical use, the proteins of the present invention can be administered orally, rectally, parenterally (particularly intravenous or subcutaneous), intracistemally, intravaginally, intraperitoneally, topically (as powders, ointments, drops or transdermal patch) bucally, or as a pulmonary or nasal inhalant. Intravenous administration will be by bolus injection or infusion over a typical period of one to several hours. In general, pharmaceutical formulations will include a ZSNK10, ZSNK11, and ZSNK12 protein, alone, or in conjunction with a dimeric partner, in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of ZSNK10, ZSNK11, and ZSNK12 is an amount sufficient to produce a clinically significant change in extracellular matrix remodeling, scar tissue formation, tumor suppression, platelet aggregation, apoptosis, and/or myogenesis.

The invention is further illustrated by the following non-limiting examples.

### EXAMPLES

From the foregoing, it will be appreciated that, although specific embodiments of the invention have been described herein for purposes of illustration, various modifications may be made without deviating from the spirit and scope of the invention. Accordingly, the invention is not limited except as by the appended claims.

### Example 1

### Chromosomal Assignment of human ZSNK10, ZSNK11, and ZSNK12

Huamn ZSNK10, ZSNK11, and ZSNK12 can be mapped using the commercially available version of the Stanford G3 Radiation Hybrid, Mapping Panel (Research Genetics, Inc., Huntsville, Ala.). The Stanford G3 RH Panel contains PCRable DNAs from each of 83 radiation hybrid clones of the whole human genome, plus two control DNAs (the RM donor and the A3 recipient). A publicly available WWW server (http://shgc-www.stanford.edu) allows chromosomal localization of markers.

With the “Stanford G3 RH Panel”, 20 μl reactions are set up in a PCRable 96-well microtiter plate (Stratagene, La Jolla, Calif.) and used in a RoboCycler Gradient 96 thermal cycler (Stratagene). Each of the 85 PCR reactions consists of 2 μl KlenTaq PCR reaction buffer (CLONTECH Laboratories, Inc., Palo Alto, Calif.), 1.6 μl dNTPs mix (2.5 mM each, PERKIN-ELMER, Foster City, Calif.), 1 μl sense primer, 1 μl antisense primer, 2 μl RediLoad (Research Genetics, Inc., Huntsville, Ala.), 0.4 μl 50× Advantage KlenTaq Polymerase Mix (CLONTECH Laboratories, Inc., Palo Alto, Calif.), 25 ng of DNA from an individual hybrid clone or control and ddH20 to make a total volume of 20 μl. The reactions are overlaid with an equal amount of mineral oil and sealed. The PCR cycler conditions are as follows: an initial 1 cycle 5 minute denaturation at 94° C., 35 cycles of a 45 seconds denaturation at 94° C., 45 seconds annealing at 70° C. and 1 minute and 15 seconds extension at 72° C., followed by a final 1 cycle extension of 7 minutes at 72° C. The reactions are separated by electrophoresis on a 2% agarose gel (Life Technologies, Gaithersburg, Md.).

### Example 2

### Synthesis of Peptides

A peptide corresponding to amino acid residue 206 (Cys) to amino acid residue 219 (Cys) of SEQ ID NO: 2, is synthesized by solid phase peptide synthesis using a model 431 A Peptide Synthesizer (Applied Biosystems/Perkin Elmer, Foster City, Calif.). Fmoc-Glutamine resin (0.63 mmol/g; Advanced Chemtech, Louisville, Ky.) is used as the initial support resin. 1 mmol amino acid cartridges (Anaspec, Inc. San Jose, Calif.) are used for synthesis. A mixture of 2(1-Hbenzotriazol-y-yl1,1,3,3-tetrahmethylhyluronium hexafluorophosphate (HBTU), 1-hydroxybenzotriazol (HOBt), 2 m N,N-Diisolpropylethylamine, N-Methylpyrrolidone, Dichloromethane (all from Applied Biosystems/Perkin Elmer) and piperidine (Aldrich Chemical Co., St. Louis, Mo.), are used for synthesis reagents.

The Peptide Companion software (Peptides International, Louisville, Ky.) is used to predict the aggregation potential and difficulty level for synthesis for the zdint-1 peptide. Synthesis is performed using single coupling programs, according to the manufacturer's specifications.

The peptide is cleaved from the solid phase following standard TFA cleavage procedure (according to Peptide Cleavage manual, Applied Biosystems/Perkin Elmer). Purification of the peptide is done by RP-HPLC using a C18, 10 μm semi-peparative column (Vydac, Hesperial, Calif.). Eluted fractions from the column are collected and analyzed for correct mass and purity by electrospray mass spectrometry. Pools of the eluted material are collected. If pure, the pools are combined, frozen and lyophilized.

The same process is repeated for the peptide corresponding to residues 461 to 747 of SEQ ID NO:17, residues 107 to 119 of SEQ ID NO:8, and/or residues 85 to 97 of SEQ ID NO:23.

### Example 3

### Anticoagulant Activity of ZSNK10, ZSNK11, and ZSNK12

The ability of the ZSNK10, ZSNK11, and ZSNK12 protein to inhibit clotting is measured in a one-stage clotting assay using wild-type ZSNK10, ZSNK11, and ZSNK12 as a control. Recombinant proteins are prepared essentially as described above from cells cultured in media containing 5 mg/ml vitamin K. Varying amounts of the ZSNK10, ZSNK11, and ZSNK12 or recombinant wild-type ZSNK10, ZSNK11, and ZSNK12 are diluted in 50 mM Tris pH 7.5, 0.1% BSA to 100 ml. The mixtures are incubated with 100 ml of ZSNK10, ZSNK11, and ZSNK12-deficient plasma and 200 ml of thromboplastin C (Dade, Miami, Fla.; contains rabbit brain thromboplastin and 11.8 mM Ca⁺⁺). The clotting assay is performed in an automatic coagulation timer (MLA Electra 800, Medical Laboratory Automation Inc., Pleasantville, N.Y.), and clotting times are converted to units of ZSNK10, ZSNK11, and ZSNK12 activity using a standard curve constructed with 1:5 to 1:640 dilutions of normal pooled human plasma (assumed to contain one unit per ml ZSNK10, ZSNK11, and ZSNK12 activity; prepared by pooling citrated serum from healthy donors).

ZSNK10, ZSNK11, and ZSNK12 activity is seen as a reduction in clotting time over control samples.

### Example 4

### Inhibition of Platelet Accumulation with ZSNK10, ZSNK11 and ZSNK12

ZSNK10, ZSNK11, and ZSNK12 is analyzed for its ability to inhibit platelet accumulation at sites of arterial thrombosis due to mechanical injury in non-human primates. A model of aortic endarterectomy is utilized in baboons, essentially as described by Lumsden et al. (Blood 81: 1762-1770 (1993)). A section of baboon aorta 1-2 cm in length is removed, inverted and scraped to remove the intima of the artery and approximately 50% of the media. The artery is reverted back to its correct orientation, cannulated on both ends and placed into an extracorporeal shunt in a baboon, thereby exposing the mechanically injured artery to baboon blood via the shunt. Just prior to opening of the shunt to the circulating blood, ¹¹¹In-labeled autologous platelets are injected intravenously into the animal. The level of platelet accumulation at the site of the injured artery is determined by real-time gamma camera imaging.

Evaluation of ZSNK10, ZSNK11, and ZSNK12 for inhibition of platelet accumulation is done using bolus injections of ZSNK10, ZSNK11, and ZSNK12 or saline control and are given just prior to the opening of the shunt. The injured arteries are measured continuously for 60 minutes.

ZSNK10, ZSNK11, and ZSNK12 activity is seen as an inhibition of platelet accumulation.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide molecule comprising the amino acid sequence as shown in SEQ ID NO:17 from residue 461 to residue 474.

2. The isolated polypeptide molecule according to claim 1, wherein the polypeptide molecule comprises the amino acid sequence from residue 398 to residue 492 as shown in SEQ ID NO:17.

3. The isolated polypeptide molecule according to claim 2, wherein the polypeptide molecule comprises the amino acid sequence from residue 191 to residue 492 as shown in SEQ ID NO:17.

4. The isolated polypeptide molecule according to claim 3, wherein the polypeptide molecule comprises the amino acid sequence from residue 1 to residue 611 as shown in SEQ ID NO:17

5. An isolated polynucleotide molecule encoding the polypeptide molecule of claim 1.

6. An isolated polypeptide molecule comprising the amino acid sequence as shown in SEQ ID NO:17 from residue 398 to residue 492.

7. An isolated polynucleotide molecule encoding the polypeptide molecule of claim 6.

8. An isolated polypeptide molecule comprising the amino acid sequence as shown in SEQ ID NO:17 from residue 191 to residue 395.

9. An isolated polynucleotide molecule encoding the polypeptide molecule of claim 8.

10. An expression vector comprising the following operably linked elements:
a) a transcription promoter;
b) a DNA segment encoding the polypeptide molecule according to claim 2; and
c) a transcription terminator.

11. The expression vector according to claim 10, wherein the DNA segment further encodes an affinity tag.

12. A cultured cell into which has been introduced an expression vector according to claim 10, wherein said cell expresses the polypeptide encoded by the DNA segment.

13. A method of producing a polypeptide comprising culturing a cell according to claim 12, whereby said cell expresses the polypeptide encoded by the DNA segment, and recovering the polypeptide.

14. The polypeptide produced by the method according to claim 13.

15. A method of producing an antibody comprising the following steps:
inoculating an animal with a polypeptide such that the polypeptide elicits an immune response in the animal to produce the antibody; and
isolating the antibody from the animal; and
wherein the polypeptide comprises at least 15 consecutive amino acids of SEQ ID NO:17.

16. The antibody produced by the method of claim 15, wherein the antibody binds to the polypeptide as shown in SEQ ID NO:17.

17. An antibody which specifically binds to a polypeptide comprising amino acid residues 1 to 611 of SEQ ID NO:17.

18. An isolated polypeptide molecule comprising the amino acid sequence as shown in SEQ ID NO:23 from residue 85 to residue 97.

19. The isolated polypeptide molecule according to claim 18, wherein the polypeptide molecule comprises the amino acid sequence from residue 51 to residue 110 as shown in SEQ ID NO:23.

20. The isolated polypeptide molecule according to claim 19, wherein the polypeptide molecule comprises the amino acid sequence from residue 1 to residue 110 as shown in SEQ ID NO:23.

21. An isolated polynucleotide molecule encoding the polypeptide molecule of claim 18.

22. An expression vector comprising the following operably linked elements:
a) a transcription promoter;
b) a DNA segment encoding the polypeptide molecule according to claim 19; and
c) a transcription terminator.

23. The expression vector according to claim 22, wherein the DNA segment further encodes an affinity tag.

24. A cultured cell into which has been introduced an expression vector according to claim 22, wherein said cell expresses the polypeptide encoded by the DNA segment.

25. A method of producing a polypeptide comprising culturing a cell according to claim 24, whereby said cell expresses the polypeptide encoded by the DNA segment, and recovering the polypeptide.

26. The polypeptide produced by the method according to claim 25.

27. A method of producing an antibody comprising the following steps:
inoculating an animal with a polypeptide such that the polypeptide elicits an immune response in the animal to produce the antibody; and
isolating the antibody from the animal; and
wherein the polypeptide comprises at least 15 consecutive amino acids of SEQ ID NO:23.

28. The antibody produced by the method of claim 27, wherein the antibody binds to the polypeptide as shown in SEQ ID NO:23.

29. An antibody which specifically binds to a polypeptide comprising amino acid residues 1 to 110 of SEQ ID NO:23.

30. An isolated polypeptide molecule comprising the amino acid sequence as shown in SEQ ID NO:20 from residue 191 to residue 393.

31. The isolated polypeptide molecule according to claim 30, wherein the polypeptide molecule comprises the amino acid sequence from residue 1 to residue 433 as shown in SEQ ID NO:20.

32. An isolated polynucleotide molecule encoding the polypeptide molecule of claim 30.

33. An expression vector comprising the following operably linked elements:
a) a transcription promoter;
b) a DNA segment encoding the polypeptide molecule according to claim 30; and
c) a transcription terminator.

34. The expression vector according to claim 33, wherein the DNA segment further encodes an affinity tag.

35. A cultured cell into which has been introduced an expression vector according to claim 33, wherein said cell expresses the polypeptide encoded by the DNA segment.

36. A method of producing a polypeptide comprising culturing a cell according to claim 35, whereby said cell expresses the polypeptide encoded by the DNA segment, and recovering the polypeptide.

37. The polypeptide produced by the method according to claim 36.

38. A method of producing an antibody comprising the following steps:
inoculating an animal with a polypeptide such that the polypeptide elicits an immune response in the animal to produce the antibody; and
isolating the antibody from the animal; and
wherein the polypeptide comprises at least 15 consecutive amino acids of SEQ ID NO:20.

39. The antibody produced by the method of claim 38, wherein the antibody binds to the polypeptide as shown in SEQ ID NO:20.

40. An antibody which specifically binds to a polypeptide comprising amino acid residues 1 to 433 of SEQ ID NO:20.

41. A method of modulating cell-cell interactions comprising contacting the cells with a polypeptide chosen from the group consisting of:
a) a polypeptide comprising the amino acid sequence as shown in SEQ ID NO:17 from residue 461 to residue 474; and
b) a polypeptide comprising the amino acid sequence as shown in SEQ ID NO:23 from residue 85 to residue 97.

42. A method of modulating cell-cell interactions comprising contacting the cells with an antibody, wherein the antibody specifically binds to a polypeptide chosen from the group consisting of:
a) a polypeptide comprising the amino acid sequence as shown in SEQ ID NO:17 from residue 1 to residue 474;
b) a polypeptide comprising the amino acid sequence as shown in SEQ ID NO:20 from residue 1 to residue 433; and
c) a polypeptide comprising the amino acid sequence as shown in SEQ ID NO:23 from residue 85 to residue 97.


SEQUENCE LISTING (24 sequences)

SEQ ID NO: 1 - DNA, 1213 bp, Sistrusus miliarius
  CDS (3)...(1211)
gagatttgattaatgtgtcatcagcagcaggtgatactttgggctcatttggagaatggagagagacagatttgctgaggcacaaaagtcatgataatgctcagttactcacgaccactgacttcgatggagacactgtaggattggcttatataagcagcatgtgccaaccgagcagttctgtaggagttattcaggaacatagcacaacaaatctcttgatggcagttacaatggcccatgagatgggtcataatctgggcatgagtcatgatggaaatcagtgtcattgtggtgctccctcgtgcattatggctgaaagactaagccaccaaccttccacacagttcagcgattgtagtgaggaatattgtcggacgtatcttaaaaatcgtagaccacaatgcattctcaatgaacccttgctgacagatattgtttcacctccagtttgtggaaatgaacttttggaggagggagaagaatgtgactgtggctctcctgcaaactgtcagaatccatgctgtgatgctgcaacgtgtaaactgacaccagggtcacagtgtgcaaaaggactgtgttgtgaccagtgcagatttaagggggcaggaacagaatgccgggcagcaaaggatgactgtgacatggctgatctctgcactggccaatctgctaagtgtcccacggatcgcttccaaaggaatggacacccatgcctaaacaacaaaggttactgctacaatcggacgtgccccaccatgaagaaccaatgtatttctttctttgggccaagtgcaactgtggctaaagattcatgtttcaaaactaaccagaaaggcagtagttatggctactgcagaaaggaaaatggtacaaagattccatgtgaaccacaagatgtaaaatgtggcaggttattctgctaccctaataaacccggaaagaagaataattgcaatgtgatatatacacccacagatgaagatattgggatggttcttcctggaacaaaatgtggacgtggaaaggtctgcagcaacgggcattgtgttgatgtggctacagcctactaatcaaccactggcttctcttagatttgattctggagattcttctttcagaaggttcaacttccctcaagtccaaagagacccatctggctgcatcctactaataaatcacccttagcttccagatggcatccaaatatgca

SEQ ID NO: 2 - protein, 400 aa, Sistrusus miliarius
DLINVSSAAGDTLGSFGEWRETDLLRHKSHDNAQLLTTTDFDGDTVGLAYISSMCQPSSSVGVIQEHSTTNLLMAVTMAHEMGHNLGMSHDGNQCHCGAPSCIMAERLSHQPSTQFSDCSEEYCRTYLKNRRPQCILNEPLLTDIVSPPVCGNELLEEGEECDCGSPANCQNPCCDAATCKLTPGSQCAKGLCCDQCRFKGAGTECRAAKDDCDMADLCTGQSAKCPTDRFQRNGHPCLNNKGYCYNRTCPTMKNQCISFFGPSATVAKDSCFKTNQKGSSYGYCRKENGTKIPCEPQDVKCGRLFCYPNKPGKKNNCNVIYTPTDEDIGMVLPGTKCGRGKVCSNGHCVDVATAYSTTGFSIFWRFFFQKVQLPSSPKRPIWLHPTNKSPLASRWHPNM

SEQ ID NO: 3 - DNA, 1200 bp, Artificial Sequence
  Degenerate polynucleotide sequence
  misc_feature 6, 15, 18, 21, 24, 27, 30, 36, 39, 42, 45, 51, 60, 66,72, 75, 78, 87, 99, 105, 108, 111, 114, 117, 129, 135, 138, 141,144, 147, 156, 159, 171, 174, 177, 180, 183, 186, 189, 204, 207,210, 216, 219, 225, 228, 231, 237, 249, 258, 261: n = A,T,C or G
  misc_feature 267, 276, 294, 297, 300, 303, 315, 321, 324, 327, 336,339, 342, 351, 360, 375, 378, 384, 393, 396, 399, 411, 420, 423,426, 429, 438, 441, 444, 447, 450, 456, 465, 468, 477, 495, 498,501, 504, 519, 531, 534, 537, 546, 549, 552, 555: n = A,T,C or G
  misc_feature 558, 567, 573, 576, 594, 603, 606, 609, 612, 621, 624,627, 648, 654, 660, 663, 669, 672, 681, 684, 690, 699, 705, 711,717, 729, 744, 747, 753, 756, 777, 786, 789, 792, 795, 798, 801,804, 813, 825, 837, 840, 843, 849, 858, 870, 873: n = A,T,C or G
  misc_feature 882, 891, 900, 909, 912, 915, 927, 936, 939, 960, 969,972, 975, 990, 996, 999, 1002, 1005, 1008, 1017, 1020, 1023,1029,1035, 1041, 1050, 1056, 1059, 1062, 1065, 1071, 1074, 1077, 1080,1086, 1098, 1116, 1122, 1125, 1128, 1131, 1134: n = A,T,C or G
  misc_feature 1140, 1143, 1152, 1158, 1161, 1170, 1173, 1176, 1179,1182,1185, 1194: n = A,T,C or G
gayytnathaaygtnwsnwsngcngcnggngayacnytnggnwsnttyggngartggmgngaracngayytnytnmgncayaarwsncaygayaaygcncarytnytnacnacnacngayttygayggngayacngtnggnytngcntayathwsnwsnatgtgycarccnwsnwsnwsngtnggngtnathcargarcaywsnacnacnaayytnytnatggcngtnacnatggcncaygaratgggncayaayytnggnatgwsncaygayggnaaycartgycaytgyggngcnccnwsntgyathatggcngarmgnytnwsncaycarccnwsnacncarttywsngaytgywsngargartaytgymgnacntayytnaaraaymgnmgnccncartgyathytnaaygarccnytnytnacngayathgtnwsnccnccngtntgyggnaaygarytnytngargarggngargartgygaytgyggnwsnccngcnaaytgycaraayccntgytgygaygcngcnacntgyaarytnacnccnggnwsncartgygcnaarggnytntgytgygaycartgymgnttyaarggngcnggnacngartgymgngcngcnaargaygaytgygayatggcngayytntgyacnggncarwsngcnaartgyccnacngaymgnttycarmgnaayggncayccntgyytnaayaayaarggntaytgytayaaymgnacntgyccnacnatgaaraaycartgyathwsnttyttyggnccnwsngcnacngtngcnaargaywsntgyttyaaracnaaycaraarggnwsnwsntayggntaytgymgnaargaraayggnacnaarathccntgygarccncargaygtnaartgyggnmgnytnttytgytayccnaayaarccnggnaaraaraayaaytgyaaygtnathtayacnccnacngaygargayathggnatggtnytnccnggnacnaartgyggnmgnggnaargtntgywsnaayggncaytgygtngaygtngcnacngcntaywsnacnacnggnttywsnathttytggmgnttyttyttycaraargtncarytnccnwsnwsnccnaarmgnccnathtggytncayccnacnaayaarwsnccnytngcnwsnmgntggcayccnaayatg

SEQ ID NO: 4 - DNA, 803 bp, Agkistrodon psicivoris
  CDS (2)...(802)
ggcccccaaaatgtgtggggtaacccagaattgggaatcatatgagcccatcacagaggcctctcagttaaatcttaatcctcaacaacaaagatataacccctacaaatacattgagcttttcctagttgtggacaacagaatggtcacaaaatacaatggcgatttagataagataaaaacaagaatatatgaacttgtcaacattttaaatgagatttacagagctttgtacattcgtgttgcactggttggcatagaattttggtgcaacagagatttgattaatgtgaaatcagcatcaggtgttactttgaaatcatttgcaaactggagagagacagtcttgccgaatcgcacaagtcatgataatgcccagttactcacggccattgtgttcgatagaggagttataggaagtgcttacccagccggcatgtgcgacctgaggcgttctgtaggaactgtccaggatcatagcgaaataaatcttcaggttgcaattacaatggcccatgagataggtcataatctgggcatgggtcatgacaataattcctgtacttgtggtggatactcatgcattatgttgcccaggttaagcgaccaaccttccaaatttttcagcaattgtagttatatccaatatcgggactttattatgaatcagaacccacaatgcattctcaatgaaccctcgggaacagatattgtttcacctccagtttgtggaaatgatattttggaggtgggagaagaatgtgactgtggctgtcctagaaattgtcaagatccatgctgca

SEQ ID NO: 5 - protein, 267 aa, Agkistrodon psicivoris
APKMCGVTQNWESYEPITEASQLNLNPQQQRYNPYKYIELFLVVDNRMVTKYNGDLDKIKTRIYELVNILNEIYRALYIRVALVGIEFWCNRDLINVKSASGVTLKSFANWRETVLPNRTSHDNAQLLTAIVFDRGVIGSAYPAGMCDLRRSVGTVQDHSEINLQVAITMAHEIGHNLGMGHDNNSCTCGGYSCIMLPRLSDQPSKFFSNCSYIQYRDFIMNQNPQCILNEPSGTDIVSPPVCGNDILEVGEECDCGCPRNCQDPCC

SEQ ID NO: 6 - DNA, 801 bp, Artificial Sequence
  Degenerate polynucleotide sequence
  misc_feature 3, 6, 18, 21, 24, 39, 48, 54, 60, 63, 69, 75, 81, 93,102, 120, 126, 129, 132, 141, 147, 150, 162, 168, 183, 186, 198,201, 210, 225, 228, 231, 240, 243, 246, 249, 252, 255, 276, 282,291, 297, 300, 303, 306, 309, 312, 315, 321, 327: n = A,T,C or G
  misc_feature 336, 342, 345, 348, 351, 357, 360, 363, 375, 381, 384,387, 390, 396, 405, 408, 411, 417, 420, 423, 429, 432, 435, 447,450, 453, 456, 459, 462, 465, 468, 480, 492, 498, 501, 507, 513,525, 534, 537, 543, 558, 564, 570, 573, 579, 591: n = A,T,C or G
  misc_feature 594, 597, 600, 603, 612, 615, 627, 636, 651, 675, 687,696, 699, 702, 705, 714, 717, 720, 723, 726, 732, 744, 750, 753,771, 777, 780, 795: n = A,T,C or G
gcnccnaaratgtgyggngtnacncaraaytgggarwsntaygarccnathacngargcnwsncarytnaayytnaayccncarcarcarmgntayaayccntayaartayathgarytnttyytngtngtngayaaymgnatggtnacnaartayaayggngayytngayaarathaaracnmgnathtaygarytngtnaayathytnaaygarathtaymgngcnytntayathmgngtngcnytngtnggnathgarttytggtgyaaymgngayytnathaaygtnaarwsngcnwsnggngtnacnytnaarwsnttygcnaaytggmgngaracngtnytnccnaaymgnacnwsncaygayaaygcncarytnytnacngcnathgtnttygaymgnggngtnathggnwsngcntayccngcnggnatgtgygayytnmgnmgnwsngtnggnacngtncargaycaywsngarathaayytncargtngcnathacnatggcncaygarathggncayaayytnggnatgggncaygayaayaaywsntgyacntgyggnggntaywsntgyathatgytnccnmgnytnwsngaycarccnwsnaarttyttywsnaaytgywsntayathcartaymgngayttyathatgaaycaraayccncartgyathytnaaygarccnwsnggnacngayathgtnwsnccnccngtntgyggnaaygayathytngargtnggngargartgygaytgyggntgyccnmgnaaytgycargayccntgytgy

SEQ ID NO: 7 - DNA, 598 bp, Agkistrodon psicivorus
  CDS (1)...(598)
aggaagagctacgttggcttgaaagcaggaagagattgcttgtcttccagccaaatccagcctccaaaatgatcccaagttctcttggtaactatatgcttagcagtttttccttatcaagggagctctataattctggaatctgggaacgtgaatgattatgaagtagtgtatccacgaaaaatcactccattgcccaaaggagcagttcagccaaagaatccgtgctgcgatgctgcaacctgtaaactgacaccaggttcacagtgtgcagaaggactgtgttgtgaccagtgcaaatttataaaagcaggaaaaatatgccggagagcaaggggtgataacccggattatcgctgcactggccaatctggtgactgtcccagaaaacacttctatgcctaaccaacaatggagatggaatggtctgcagcaacaggcagtgtgttgatgtgacttcaacctaataatcaacctctggcttctctcagatttgattttggagatccttcttccagaaggtttggcttccctgtagtccaaagagacccatctgcctgcatcctactagtaaatcactcttagctttcatatggaa

SEQ ID NO: 8 - protein, 195 aa, Agkistrodon psicivorus
RKSYVGLKAGRDCLSSSQIQPPKSQVLLVTICLAVFPYQGSSIILESGNVNDYEVVYPRKITPLPKGAVQPKNPCCDAATCKLTPGSQCAEGLCCDQCKFIKAGKICRRARGDNPDYRCTGQSGDCPRKHFYAPTMEMEWSAATGSVLMLQPNNQPLASLRFDFGDPSSRRFGFPVVQRDPSACILLVNHSLSYG

SEQ ID NO: 9 - DNA, 585 bp, Artificial Sequence
  Degenerate polynucleotide sequence
  misc_feature 3, 9, 15, 18, 21, 27, 30, 33, 42, 45, 48, 51, 63, 66,72, 78, 81, 84, 87, 90, 99, 102, 105, 111, 120, 123, 126, 135,141, 144, 150, 165, 168, 174, 177, 186, 189, 192, 195, 201, 204,207, 213, 222, 234, 237, 240, 249, 252, 255, 258: n = A,T,C or G
  misc_feature 261, 270, 276, 279, 309, 312, 324, 327, 330, 333, 336,345, 354, 360, 363, 369, 372, 381, 384, 399, 402, 405, 423, 426,429, 432, 435, 438, 441, 444, 450, 456, 468, 471, 474, 477, 480,483, 495, 501, 504, 507, 510, 513, 519, 525, 528: n = A,T,C or G
  misc_feature 531, 537, 543, 546, 549, 558, 561, 564, 573, 576, 579,585: n = A,T,C or G
mgnaarwsntaygtnggnytnaargcnggnmgngaytgyytnwsnwsnwsncarathcarccnccnaarwsncargtnytnytngtnacnathtgyytngcngtnttyccntaycarggnwsnwsnathathytngarwsnggnaaygtnaaygaytaygargtngtntayccnmgnaarathacnccnytnccnaarggngcngtncarccnaaraayccntgytgygaygcngcnacntgyaarytnacnccnggnwsncartgygcngarggnytntgytgygaycartgyaarttyathaargcnggnaarathtgymgnmgngcnmgnggngayaayccngaytaymgntgyacnggncarwsnggngaytgyccnmgnaarcayttytaygcnccnacnatggaratggartggwsngcngcnacnggnwsngtnytnatgytncarccnaayaaycarccnytngcnwsnytnmgnttygayttyggngayccnwsnwsnmgnmgnttyggnttyccngtngtncarmgngayccnwsngcntgyathytnytngtnaaycaywsnytnwsntayggn

SEQ ID NO: 10 - protein, 4 aa, Artificial Sequence
  Atificial peptide
MSEC

SEQ ID NO: 11 - protein, 4 aa, Artificial Sequence
  Atificial peptide
RSEC

SEQ ID NO: 12 - protein, 4 aa, Artificial Sequence
  Atificial peptide
IDDC

SEQ ID NO: 13 - protein, 4 aa, Artificial Sequence
  Atificial peptide
RDDC

SEQ ID NO: 14 - protein, 4 aa, Artificial Sequence
  Atificial peptide
  VARIANT (1)...(2): Xaa = Any amino acid
  VARIANT 1, 2: Xaa = Any Amino Acid
XXCD

SEQ ID NO: 15 - protein, 4 aa, Artificial Sequence
  Atificial peptide
DDCD

SEQ ID NO: 16 - DNA, 2334 bp, Sistrusus miliarius
  CDS (95)...(1930)
  misc_feature 2334: n = A,T,C or G
gaattcggaacgaggtagtcaacagaggaagagctcaggttggcctgaaagcaggaagtgattgcctgtcttccagccaaatccagcctccaaaatgatccaagttctcttggtgactatatgcttagcagcgtttccttatcaagggagctctataatcctggaatctgggaacgtgaatgattatgaagtagtgtatacacgaaaagtcactgcattgcccaaaggagcagctcagccaaagtatgaagacgccatgcaatatgaatttaagatgaacggagagccagtggtccttcacctggaaaaaaataaaagacttttttcaaaagattacagcgagactcattattcccctgatggcagacaaattacaacataccccatgattgaggatcactgctattatcatggacgcatccagaatgatgctgactcaactgcaagcatcagtgcatgcaacggtttgaaaggacatttcaagcttcaaggggagatgtacctcattgaacccttgaagcttcccgacagtgaagcccatgcagtctacaaatatgaaaacatagaaaaagaggatgaggcccccaaaatgtgtggggtaacccagaattgggaatcatatgagcccatcaaaaaggcctttcagttaaatcttactcctgaacaacaagcatacttggatgccaaaaaatacgttgagtttgttgtagttctggaccatggaatgtacacaaaatacaaagatgatttagataagataaaaacaagaatatatgaaattgtcaacactatgaatgagatttacatacctttgaatattcgtgtcgcactggttcacctagaaatttggtccaacagagatttgattaatgtgtcatcagcagcaggtgatactttgggctcatttggagaatggagagagacagatttgctgaggcacaaaagtcatgataatgctcagttactcacgaccactgacttcgatggagacactgtaggattggcttatataagcagcatgtgccaaccgagcagttctgtaggagttattcaggaacatagcacaacaaatctcttgatggcagttacaatggcccatgagatgggtcataatctgggcatgagtcatgatggaaatcagtgtcattgtggtgctccctcgtgcattatggctgaaagactaagccaccaaccttccacacagttcagcgattgtagtgaggaatattgtcggacgtatcttaaaaatcgtagaccacaatgcattctcaatgaacccttgctgacagatattgtttcacctccagtttgtggaaatgaacttttggaggagggagaagaatgtgactgtggctctcctgcaaactgtcagaatccatgctgtgatgctgcaacgtgtaaactgacaccagggtcacagtgtgcaaaaggactgtgttgtgaccagtgcagatttaagggggcaggaacagaatgccgggcagcaaaggatgactgtgacatggctgatctctgcactggccaatctgctaagtgtcccacggatcgctcccaaaggaatggacacccatgcctaaacaacaaaggttactgctacaatcggacgtgccccaccatgaagaaccaatgtatttctttctttgggccaagtgcaactgtggctaaagattcatgtttcaaaactaaccagaaaggcagtagttatggctactgcagaaaggaaaatggtacaaagattccatgtgaaccacaagatgtaaaatgtggcaggttattctgctaccctaataaacccggaaagaagaataattgcaatgtgatatatacacccacagatgaagatattgggatggttcttcctggaacaaaatgtggacgtggaaaggtctgcagcaacgggcattgtgttgatgtggctacagcctactaatcaaccactggcttctctcagatttgattctggagattcttctttcagaaggttcaacttccctcaagtccaaagagacccatctgcctgcatcctactaataaatcacccttagcttccagatggcatccaaattatgcaatatttaatctgtttacattttactgtaatcaaaccttttccccaccacaaagctccatgggcatgtacaacaccaaaggcttatttgctgtcaaggaaaaaaaaaatggccattttactgtttgccaattgcagagcacatttaagttctgccttttgagctggtgtattcaaagtcaatgcttcctctcccaaaatttcatgctggctttccaagatgtagctgcttccatcaataaactcactatcctcattccaaaaan

SEQ ID NO: 17 - protein, 611 aa, Sistrusus miliarius
MIQVLLVTICLAAFPYQGSSIILESGNVNDYEVVYTRKVTALPKGAAQPKYEDAMQYEFKMNGEPVVLHLEKNKRLFSKDYSETHYSPDGRQITTYPMIEDHCYYHGRIQNDADSTASISACNGLKGHFKLQGEMYLIEPLKLPDSEAHAVYKYENIEKEDEAPKMCGVTQNWESYEPIKKAFQLNLTPEQQAYLDAKKYVEFVVVLDHGMYTKYKDDLDKIKTRIYEIVNTMNEIYIPLNIRVALVHLEIWSNRDLINVSSAAGDTLGSFGEWRETDLLRHKSHDNAQLLTTTDFDGDTVGLAYISSMCQPSSSVGVIQEHSTTNLLMAVTMAHEMGHNLGMSHDGNQCHCGAPSCIMAERLSHQPSTQFSDCSEEYCRTYLKNRRPQCILNEPLLTDIVSPPVCGNELLEEGEECDCGSPANCQNPCCDAATCKLTPGSQCAKGLCCDQCRFKGAGTECRAAKDDCDMADLCTGQSAKCPTDRSQRNGHPCLNNKGYCYNRTCPTMKNQCISFFGPSATVAKDSCFKTNQKGSSYGYCRKENGTKIPCEPQDVKCGRLFCYPNKPGKKNNCNVIYTPTDEDIGMVLPGTKCGRGKVCSNGHCVDVATAY

SEQ ID NO: 18 - DNA, 1833 bp, Artificial Sequence
  Degenerate polynucleotide sequence
  misc_feature 12, 15, 18, 21, 24, 33, 36, 39, 45, 54, 57, 60, 69, 75,78, 84, 99, 102, 108, 111, 117, 120, 123, 126, 129, 135, 138, 141,147, 162, 189, 195, 198, 201, 204, 210, 225, 228, 234, 246, 252,261, 264, 270, 273, 282, 285, 291, 321, 324: n = A,T,C or G
  misc_feature 339, 345, 348, 351, 354, 360, 363, 372, 375, 381, 393,399, 411, 420, 423, 429, 432, 438, 444, 450, 453, 489, 492, 504,507, 510, 525, 534, 546, 555, 561, 564, 567, 579, 585, 591, 603,612, 615, 618, 621, 630, 639, 657, 672, 675, 690: n = A,T,C or G
  misc_feature 696, 717, 720, 729, 732, 735, 738, 741, 747, 759, 765,771, 780, 783, 786, 789, 792, 795, 801, 804, 807, 810, 816, 825,831, 837, 840, 843, 852, 864, 870, 873, 876, 879, 882, 894, 900,903, 906, 909, 912, 921, 924, 936, 939, 942, 945: n = A,T,C or G
  misc_feature 948, 951, 954, 969, 972, 975, 981, 984, 990, 993, 996,1002, 1014, 1023, 1026, 1032, 1041, 1059, 1062, 1065, 1068, 1080,1086, 1089, 1092, 1101, 1104, 1107, 1116, 1125, 1140, 1143, 1149,1158, 1161, 1164, 1176, 1185, 1188, 1191, 1194: n = A,T,C or G
  misc_feature 1203, 1206, 1209, 1212, 1215, 1221, 1230, 1233, 1242,1260, 1263, 1266, 1269, 1284, 1296, 1299, 1302, 1311, 1314, 1317,1320, 1323, 1332, 1338, 1341, 1359, 1368, 1371, 1374, 1377, 1386,1389, 1392, 1413, 1419, 1425, 1428, 1434, 1437: n = A,T,C or G
  misc_feature 1446, 1449, 1455, 1458, 1464, 1470, 1476, 1482, 1494,1509, 1512, 1518, 1521, 1542, 1551, 1554, 1557, 1560, 1563, 1566,1569, 1578, 1590, 1602, 1605, 1608, 1614, 1623, 1635, 1638, 1647,1656, 1665, 1674, 1677, 1680, 1692, 1701, 1704: n = A,T,C or G
  misc_feature 1725, 1734, 1737, 1740, 1755, 1761, 1764, 1767, 1770,1773, 1782, 1785, 1788, 1794, 1800, 1806, 1815, 1821, 1824, 1827,1830: n = A,T,C or G
atgathcargtnytnytngtnacnathtgyytngcngcnttyccntaycarggnwsnwsnathathytngarwsnggnaaygtnaaygaytaygargtngtntayacnmgnaargtnacngcnytnccnaarggngcngcncarccnaartaygargaygcnatgcartaygarttyaaratgaayggngarccngtngtnytncayytngaraaraayaarmgnytnttywsnaargaytaywsngaracncaytaywsnccngayggnmgncarathacnacntayccnatgathgargaycaytgytaytaycayggnmgnathcaraaygaygcngaywsnacngcnwsnathwsngcntgyaayggnytnaarggncayttyaarytncarggngaratgtayytnathgarccnytnaarytnccngaywsngargcncaygcngtntayaartaygaraayathgaraargargaygargcnccnaaratgtgyggngtnacncaraaytgggarwsntaygarccnathaaraargcnttycarytnaayytnacnccngarcarcargcntayytngaygcnaaraartaygtngarttygtngtngtnytngaycayggnatgtayacnaartayaargaygayytngayaarathaaracnmgnathtaygarathgtnaayacnatgaaygarathtayathccnytnaayathmgngtngcnytngtncayytngarathtggwsnaaymgngayytnathaaygtnwsnwsngcngcnggngayacnytnggnwsnttyggngartggmgngaracngayytnytnmgncayaarwsncaygayaaygcncarytnytnacnacnacngayttygayggngayacngtnggnytngcntayathwsnwsnatgtgycarccnwsnwsnwsngtnggngtnathcargarcaywsnacnacnaayytnytnatggcngtnacnatggcncaygaratgggncayaayytnggnatgwsncaygayggnaaycartgycaytgyggngcnccnwsntgyathatggcngarmgnytnwsncaycarccnwsnacncarttywsngaytgywsngargartaytgymgnacntayytnaaraaymgnmgnccncartgyathytnaaygarccnytnytnacngayathgtnwsnccnccngtntgyggnaaygarytnytngargarggngargartgygaytgyggnwsnccngcnaaytgycaraayccntgytgygaygcngcnacntgyaarytnacnccnggnwsncartgygcnaarggnytntgytgygaycartgymgnttyaarggngcnggnacngartgymgngcngcnaargaygaytgygayatggcngayytntgyacnggncarwsngcnaartgyccnacngaymgnwsncarmgnaayggncayccntgyytnaayaayaarggntaytgytayaaymgnacntgyccnacnatgaaraaycartgyathwsnttyttyggnccnwsngcnacngtngcnaargaywsntgyttyaaracnaaycaraarggnwsnwsntayggntaytgymgnaargaraayggnacnaarathccntgygarccncargaygtnaartgyggnmgnytnttytgytayccnaayaarccnggnaaraaraayaaytgyaaygtnathtayacnccnacngaygargayathggnatggtnytnccnggnacnaartgyggnmgnggnaargtntgywsnaayggncaytgygtngaygtngcnacngcntay

SEQ ID NO: 19 - DNA, 1812 bp, Agkistrodon piscivorus
  CDS (93)...(1397)
  misc_feature 1812: n = A,T,C or G
gaattcggcacgagggtcaacagaggaagagctcaggttggcttgaaagaaggaagagattgcctgtcttccagccaaatccagcctccaaaatgatccaggttctcttggtgactatatgcttagcagcttttccttatcaagggagctctataatcctggaatctgggaacgttaatgattatgaaatagtgtatccacgaaaagtcactccagtgcccagaggagcagttcagccaaagtatgaagatgccatgcaatatgaattgaaagtgaatggagagccagtggtccttcacctggaaaaaaataaaggacttttttcagaagattacagcgagactcattattcccctgatggcagagaaattacaacataccccctggttgaggatcactgctattatcatggacgcatcgagaatgatgctgactcaactgcaagcatcagtacatgcaacggtttgaaaggacatttcaagcttcaaggggagatgtaccttattgaaccgttggagctttctgacagtgaagcccatgcagtcttcaaatatgaaaatgtagaaaaagaggatgaggcccccaaaatgtgtggggtaacccagaattgggaatcatatgagcccatcaaaaaggcctttcagttaaatcttactcctgaacaacaaggattcccccaaagatatgttgagcttgtcatagttgcagatcacagaatgaacacgaaatacaaaggtgattcagataagataagacaatgggtacatcaaattgtcaacactataaatgagatttacagacctttgaatattcaatttgcactggttggcctagaaatttggtccaaccaagatttgattaccgtgacgtcagtatcagatgatactttggcctcatttgcaaactggagacagacagatttgctgaatcgcataagtcatgataatgcccagttactcacggccattgacttcgatggagacactgtaggattggcttatgtgggcggcatgtgccaactgaagcattctacaggagttgtccaggatcatagtgcaataaatcttttggttgcagttacaatggcccatgagctgggtcataatctgggcatgaatcatgatgaaaatcagtgtcattgcggtgctaactcgtgcgttatggctgacacactaagtgatcaaccttccaaactattcagcgattgtagtaagaaagactatcagacgtttcttacggttaaaaacccacaatgcattctcaataaacccttgagaacagatactgtttcaactccagtttctggaaatgaactttgggaggcgggagaagaatgtgactgtggctctcctagagtctgcagcaacaggcagtgtgttgatgtgactacagcctaataatcaacctctggcttctctcagatttgatcttggagatccttcttccagaaggtttcacttccctcaaatccaaagagatccatctgcctgcatcctactagtaaatcactcttagctttcatatggaatctaacttctgcaatatttcttctccatatttaatctgtttaccttttgctgtaatcaaaccttttcccaccacaaagctctatgggcatgtacaacaccaacggcttatctgctgtcaagaaaaaaaatggccattttaccgtttgccaaagcacatttaatgcaacaagttctgccttttgagctggtgtattcgaagtgaatgtttactctcccaaaatttcatgctggctttccaagatgtagctgcttccgtcaataaactaactattctcattcaaan

SEQ ID NO: 20 - protein, 434 aa, Agkistrodon piscivorus
MIQVLLVTICLAAFPYQGSSIILESGNVNDYEIVYPRKVTPVPRGAVQPKYEDAMQYELKVNGEPVVLHLEKNKGLFSEDYSETHYSPDGREITTYPLVEDHCYYHGRIENDADSTASISTCNGLKGHFKLQGEMYLIEPLELSDSEAHAVFKYENVEKEDEAPKMCGVTQNWESYEPIKKAFQLNLTPEQQGFPQRYVELVIVADHRMNTKYKGDSDKIRQWVHQIVNTINEIYRPLNIQFALVGLEIWSNQDLITVTSVSDDTLASFANWRQTDLLNRISHDNAQLLTAIDFDGDTVGLAYVGGMCQLKHSTGVVQDHSAINLLVAVTMAHELGHNLGMNHDENQCHCGANSCVMADTLSDQPSKLFSDCSKKDYQTFLTVKNPQCILNKPLRTDTVSTPVSGNELWEAGEECDCGSPRVCSNRQCVDVTTA

SEQ ID NO: 21 - DNA, 1833 bp, Artificial Sequence; identical to SEQ ID NO: 18
  Degenerate polynucleotide sequence
  misc_feature 12, 15, 18, 21, 24, 33, 36, 39, 45, 54, 57, 60, 69, 75,78, 84, 99, 102, 108, 111, 117, 120, 123, 126, 129, 135, 138, 141,147, 162, 189, 195, 198, 201, 204, 210, 225, 228, 234, 246, 252,261, 264, 270, 273, 282, 285, 291, 321, 324: n = A,T,C or G
  misc_feature 339, 345, 348, 351, 354, 360, 363, 372, 375, 381, 393,399, 411, 420, 423, 429, 432, 438, 444, 450, 453, 489, 492, 504,507, 510, 525, 534, 546, 555, 561, 564, 567, 579, 585, 591, 603,612, 615, 618, 621, 630, 639, 657, 672, 675, 690: n = A,T,C or G
  misc_feature 696, 717, 720, 729, 732, 735, 738, 741, 747, 759, 765,771, 780, 783, 786, 789, 792, 795, 801, 804, 807, 810, 816, 825,831, 837, 840, 843, 852, 864, 870, 873, 876, 879, 882, 894, 900,903, 906, 909, 912, 921, 924, 936, 939, 942, 945: n = A,T,C or G
  misc_feature 948, 951, 954, 969, 972, 975, 981, 984, 990, 993, 996,1002, 1014, 1023, 1026, 1032, 1041, 1059, 1062, 1065, 1068, 1080,1086, 1089, 1092, 1101, 1104, 1107, 1116, 1125, 1140, 1143, 1149,1158, 1161, 1164, 1176, 1185, 1188, 1191, 1194: n = A,T,C or G
  misc_feature 1203, 1206, 1209, 1212, 1215, 1221, 1230, 1233, 1242,1260, 1263, 1266, 1269, 1284, 1296, 1299, 1302, 1311, 1314, 1317,1320, 1323, 1332, 1338, 1341, 1359, 1368, 1371, 1374, 1377, 1386,1389, 1392, 1413, 1419, 1425, 1428, 1434, 1437: n = A,T,C or G
  misc_feature 1446, 1449, 1455, 1458, 1464, 1470, 1476, 1482, 1494,1509, 1512, 1518, 1521, 1542, 1551, 1554, 1557, 1560, 1563, 1566,1569, 1578, 1590, 1602, 1605, 1608, 1614, 1623, 1635, 1638, 1647,1656, 1665, 1674, 1677, 1680, 1692, 1701, 1704: n = A,T,C or G
  misc_feature 1725, 1734, 1737, 1740, 1755, 1761, 1764, 1767, 1770,1773, 1782, 1785, 1788, 1794, 1800, 1806, 1815, 1821, 1824, 1827,1830: n = A,T,C or G

SEQ ID NO: 22 - DNA, 908 bp, Agkistrodon piscivorus
  CDS (96)...(429)
  misc_feature 908: n = A,T,C or G
gaattcggcacgaggataatcaacagaggaagagctcacgttggcttgaaagcaggaagagattgcttgtcttccagccaaatccagcctccaaaatgatccaagttctcttggtaactatatgcttagcagtttttccttatcaagggagctctataattctggaatctgggaacgtgaatgattatgaagtagtgtatccacgaaaaatcactccattgcccaaaggagcagttcagccaaagaatccgtgctgcgatgctgcaacctgtaaactgacaccaggttcacagtgtgcagaaggactgtgttgtgaccagtgcaaatttataaaagcaggaaaaatatgccggagagcaaggggtgataacccggattatcgctgcacaggccaatctggtgactgtcccagaaaacacttctatgcctaaccaacaatggagatggaatggtctgcagcaacaggcagtgtgttgatgtgacttcagcctaataatcaacctctggcttctctcagatttgattttggagatccttcttccagaaggtttggcttccctgtagtccaaagagacccatctgcctgcatcctactagtaaatcactcttagctttcatatggaatctaacttctgcaatatttcttctccatatttaatctgtttaccttttgctgtaatcaaaccttttcccaccacaaagctctatgggcatgtacaacaccaacggcttatctgctgtcaagaaaaaaaatggccattttaccgtttgccaaagcacatttaatgcaacaagttctgccttttgagctggtgtattcgaagtgaatgtttactctcccaaaatttcatgctggctttccaagatgtagctgcttccgtcaataaactaactattctcattcaaan

SEQ ID NO: 23 - protein, 111 aa, Agkistrodon piscivorus
MIQVLLVTICLAVFPYQGSSIILESGNVNDYEVVYPRKITPLPKGAVQPKNPCCDAATCKLTPGSQCAEGLCCDQCKFIKAGKICRRARGDNPDYRCTGQSGDCPRKHFYA

SEQ ID NO: 24 - DNA, 333 bp, Artificial Sequence
  Degenerate polynucleotide sequence
  misc_feature 12, 15, 18, 21, 24, 33, 36, 39, 45, 54, 57, 60, 69, 75,78, 84, 99, 102, 108, 111, 120, 123, 126, 129, 135, 138, 141, 147,156, 168, 171, 174, 183, 186, 189, 192, 195, 204, 210, 213, 243,246, 258, 261, 264, 267, 270, 279, 288, 294: n = A,T,C or G
  misc_feature 297, 303, 306, 315, 318, 333: n = A,T,C or G
atgathcargtnytnytngtnacnathtgyytngcngtnttyccntaycarggnwsnwsnathathytngarwsnggnaaygtnaaygaytaygargtngtntayccnmgnaarathacnccnytnccnaarggngcngtncarccnaaraayccntgytgygaygcngcnacntgyaarytnacnccnggnwsncartgygcngarggnytntgytgygaycartgyaarttyathaargcnggnaarathtgymgnmgngcnmgnggngayaayccngaytaymgntgyacnggncarwsnggngaytgyccnmgnaarcayttytaygcn